(19) United States
(12) Patent Application Publication
(10) Pub. No.: US 2005/0064527 A1
(43) Pub. Date: Mar. 24, 2005

(54) NIMR compositions and their methods of use

(76) Inventors: Stuart B. Levy, Boston, MA (US); Teresa M. Barbosa, S. Pedro da Cova (PT); Michael N. Alekshun, Wakefield, MA (US)

Correspondence Address: LAHIVE & COCKFIELD, LLP., 28 STATE STREET, BOSTON, MA, 02109, US

(21) Appl. No.: 10/893,671

(22) Filed: Jul. 15, 2004

Related U.S. Application Data
(63) Continuation of application No. 09/801,563, filed on Mar. 8, 2001, now abandoned.
(60) Provisional application No. 60/188,362, filed on Mar. 10, 2000.

(51) Int. Cl. G01N 33/554; G01N 33/569; C12Q 1/04

(52) U.S. Cl. 435/7.32; 435/34

(57) ABSTRACT

Newly identified mar regulated (NIMR) genes and polypeptides are described. In addition, screening assays to identify agents that modulate NIMR activity are provided.

### RELATED APPLICATION INFORMATION

This application claims priority to U.S. Ser. No. 60/188,362, filed Mar. 10, 2000. The entire contents of this application are hereby incorporated by reference.

### GOVERNMENT FUNDING

This work was funded, in part, by United States Public Health Service Grant number GM51661. The government may, therefore, have certain rights in this invention.

### BACKGROUND OF THE INVENTION

Multidrug resistance in microbes is generally attributed to the acquisition of multiple transposons and plasmids bearing genetic determinants for different mechanisms of resistance (Gold et al. 1996. N. Engl. J. Med. 335:1445). However, descriptions of intrinsic mechanisms that confer multidrug resistance have begun to emerge. The first of these was a chromosomally encoded multiple antibiotic resistance (mar) locus in Escherichia coli (George and Levy. 1983. J. Bacteriol. 155:531; George and Levy 1983. J. Bacteriol. 155:541).

The multiple antibiotic resistance (mar) locus is a chromosomally encoded locus that controls an adaptational response to antibiotics and other environmental hazards (Alekshun, M. N. and Levy, S. B. 1997. Antimicrob. Agents Chemother. 10: 2067-2075). The mar locus consists of two divergently positioned transcriptional units that flank a common promoter/operator region in E. coli and Salmonella typhimurium (Alekshun and Levy. 1997. Antimicrobial Agents and Chemother. 41: 2067) and Shigella flexneri (Barbosa and Levy. 1999. 99^{th} General Meeting of the American Society for Microbiology (Chicago, Ill.). Abstract A42, p. 9). One unit encodes MarC, a putative integral inner membrane polypeptide without any yet apparent function, but which appears to contribute to the Mar phenotype in some strains. The other unit-comprises the marRAB operon, encoding the Mar repressor (MarR), which binds marO and negatively regulates expression of marRAB (Cohen et al. 1994. J. Bacteriol. 175:1484; Martin and Rosner. 1995. Proc. Natl. Acad. Sci. USA 92:5456; Seoane and Levy. 1995. J. Bacteriol. 177:530), an activator (MarA), which activates expression of MarRAB and controls expression of other genes on the chromosome, i.e., the mar regulon (Cohen et al. 1994. J. Bacteriol. 175:1484; Gambino et. al. 1993. J. Bacteriol. 175:2888; Seoane and Levy. 1995. J. Bacteriol. 177:530), and a putative small polypeptide (MarB) of unknown function. MarA is a member of the XyIS/AraC family of transcriptional activators (Gallegos et al. 1993. Nucleic Acids Res. 21:807).

The prior art has identified the mar regulon as comprising acrAB, fumC, inaA, marA, marB, marR, ompF, ompX, sodA, tolC, and zwf. Given the role of the mar locus in controlling bacterial responses to environmental stress, identification of other genes that are regulated by MarA will be of great benefit in controlling microbes.

### SUMMARY

The present invention represents an important advance in controlling microbial adaptation to environmental stress signals by newly identifying genes which respond to high constitutive levels or to overexpression of marA and, thus, are important in mediating resistance to and survival in environmental stresses in microbial cells. Further, the instant invention identifies genes under the control of MarA as being important in regulating virulence in microbes. Accordingly, the instant invention provides novel targets (genes and polypeptides) for use in screening assays to identify compounds that modulate microbial adaptation to stress and/or virulence.

In one aspect, the invention provides a method for identifying compounds that modulate an NIMR polypeptide activity comprising:
contacting an NIMR polypeptide with a test compound under conditions which allow interaction of the compound with the polypeptide;
determining the ability of the test compound to modulate the activity of an NIMR polypeptide; and
selecting those compounds that modulate the activity of the NIMR polypeptide to thereby identify compounds that modulate NIMR polypeptide activity.

In one embodiment, the NIMR polypeptide is selected from the group consisting of: b0357, b0447, b0853, b1448, b2530, b2889, b2948, b3469, mdaB, yadG, yadH, ybjC, yfaE, yggJ, and yhbW.

In another embodiment, the NIMR polypeptide activity comprises promoting the ability of a microbe to resist an environmental challenge. In another embodiment, the NIMR polypeptide is selected from the group consisting of: aceG, ackA, aldA, cobU, fabB, fecA, galK, galT, gatA, gatC, glpD, gltA, gshB, guaB, hemB, map, mglB, mtr, ndh, nfnB, pflB, pgi, purA, ribD, rimK, rplE, srlA_{—}2, tnaA, tnaL, tpx, acnA, mdaA, ribA, andydeA.

In another embodiment, the NIMR polypeptide activity comprises promotion of microbial virulence. In oner embodiment, the NIMR polypeptide is selected from the group consisting of: aceG, ackA, aldA, cobU, fabB, fecA, galK, galT, gatA, gatC, glpD, gitA, gshB, guaB, hemB, map, mglB, mtr, ndh, nfnB, pflB, pgi, purA, ribD, rimK, rplE, srA_{—}2, tnaA, tnaL, tpx, acnA, mdaA, ribA, andydeA.

In one embodiment, the step of determining comprises measuring the efflux of the test compound or a marker compound from the cell.

In one embodiment, the step of determining comprises measuring the ability of the microbe to grow or remain viable in the presence of the environmental challenge.

In one embodiment, the NIMR polypeptide is present in a microbial cell.

In another embodiment, the NIMR polypeptide is heterologous to the cell in which it is present.

In another aspect, the invention pertains to a method for identifying compounds that modulate an NIMR polypeptide activity comprising:
contacting an NIMR polypeptide with a test compound under conditions which allow interaction of the compound with the polypeptide;
determining the ability of the test compound to modulate the expression of an NIMR polypeptide; and
selecting those compounds that modulate the expression of the NIMR polypeptide to thereby identify compounds that modulate NIMR polypeptide activity.

In one embodiment, the NIMR polypeptide is selected from the group consisting of: b0357, b0447, b0853, b1448, b2530, b2889, b2948, b3469, mdaB, yadG, yadH, ybjC, yfaE, yggJ, and yhbW.

In one embodiment, the NIMR polypeptide is selected from the group consisting of: aceG, ackA, aldA, cob U, fabB, fecA, galK, galT, gatA, gatC, glpD, gltA, gshB, guaB, hemB, map, mglB, mtr, ndh, nfnb, pflB, pgi, purA, ribD, rimK, rplE, srlA_{—}2, tnaA, tnaL, tpx, acnA, mdaA, ribA, and ydeA.

In one embodiment, the step of measuring comprises measuring the amount of RNA produced by the cell.

In one embodiment, the step of measuring comprises measuring the amount or activity of a reporter gene product produced by the cell. In another embodiment, the step of measuring comprises detecting the ability of an antibody to bind to the reporter gene product.

In one embodiment, the NIMR polypeptide is present in a cell free system.

In one embodiment, the step of determining comprises measuring the ability of the compound to bind to the NIMR polypeptide.

In one aspect, the invention pertains to a method for decreasing the virulence of a microbe comprising exposing the microbe to an environmental challenge and to an agent that modulates the activity of an NIMR polypeptide.

In another aspect, the invention pertains to a method for reducing the marA mediated transcription of an NIMR gene comprising exposing the microbe to an environmental challenge and to an agent that modulates the activity of an NIMR polypeptide.

In another aspect, the invention pertains to a method for identifying compounds that modulate activity of an NIMR polypeptide in a microbe comprising: contacting an isolated NIMR nucleic acid molecule with a test compound under conditions which allow interaction of the compound with the nucleic acid molecule; determining the ability of the test compound to bind to the isolated NIMR nucleic acid molecule; and selecting those compounds that bind to the NIMR nucleic acid molecule to thereby identify compounds that modulate activity of an NIMR polypeptide.

In one embodiment, the NIMR polypeptide is selected from the group consisting of: b0357, b0447, b0853, b1448, b2530, b2889, b2948, b3469, mdaB, yadG, yadH, ybjC, yfaE, yggJ and yhbW.

In one embodiment, the NIMR polypeptide activity comprises promoting the ability of a microbe to resist an environmental challenge.

In one embodiment, the NIMR polypeptide is selected from the group consisting of: aceG, ackA, aldA, cobU, fabB, fecA, galk, galT, gatA, gatC, glpD, gitA, gshB, guaB, hemB, map, mglB, mir, ndh, nfnB, pfiB, pgi, purA, ribD, rimK, rplE, srlA_{—}2, tnaA, tnaL, tpx, acna, mdaA, ribA, andydeA.

In another embodiment, the NIMR polypeptide activity comprises promotion of the virulence of a microbe.

In yet another embodiment, the NIMR polypeptide is selected from the group consisting of: aceG, ackA, alda, cobU, fabB, fecA, galK, galt gatA, gatC, glpD, gltA, gshB, guaB, hemB, map, mglB, mtr, ndh, nfnB, pflB, pgi, purA, ribD, rimK, rplE, srA_{—}2, tnaA, tnaL, tpx, acnA, mdaA, ribA, and ydeA.

In one embodiment, the environmental challenge is an antibiotic compound.

In another embodiment, the environmental challenge is non-antibiotic compound.

In yet another embodiment, the non-antibiotic compound is a candidate disinfectant or antiseptic compound.

In yet another aspect, the invention pertains to a vaccine comprising an NIMR nucleic acid molecule or an NIMR polypeptide and a pharmaceutically acceptable carrier.

In another aspect, the invention pertains to a composition comprising a compound that modulates the activity of an NIMR polypeptide and an antibiotic.

In still another aspect, the invention pertains ot a composition comprising a compound that modulates the activity of an NIMR polypeptide and a non-antibiotic compound.

In yet another aspect, the invention pertains to a method for reducing the virulence of a microbe in a subject suffering from a microbial infection comprising administering an NIMR modulating agent to the subject such that the virulence of the microbe is reduced.

In another aspect, the invention pertains to a method for treating a microbial infection in a subject comprising administering an NIMR modulating agent to the subject such that the infection is treated.

In another aspect, the invention pertains to a method for reducing the infectivity of a microbe on a surface comprising contacting the microbe with an NIMR modulating agent such that the infectivity of the microbe is reduced.

In one embodiment, the microbe is a gram positive bacterium. In another embodiment, the microbe is a gram negative bacterium. In still another embodiment, the microbe is an acid fast bacterium.

### BRIEF DESCRIPTION OF THE DRAWINGS

FIG. 1 illustrates a gene expression profile of the Escherichia coli MarA regulated genes.

FIG. 2 illustrates the chromosomal distribution and location of the different members of the mar regulon.

FIG. 3 illustrates northern blot analysis of NIMR genes.

### DETAILED DESCRIPTION

Although the mar regulon was previously identified as being involved in multidrug resistance, the instant invention demonstrates that many more genes of more varied function than previously taught or suggested in the art are under the control, either directly or indirectly, of marA. The present invention represents an important advance in controlling microbial adaptation to stress and/or virulence by newly identifying genes that respond to high constitutive expression or to the overexpression of marA, and referred to herein as “Newly Identified MarA Responsive (NIMR) genes.” The identification of these genes provides novel targets, both nucleic acid and polypeptide targets, for use in screening assays to identify compounds that modulate microbial responses to environmental stress and, thereby, modulate microbial adaptation to their environment and/or microbial virulence. Compounds identified in such screening assays can be used, e.g., to improve the activity of antibiotics, to improve the activity of non-antibiotic agents (e.g., disinfectants), and to prevent the MarA induced expression of NIMR genes.

Before further description of the invention, certain terms employed in the specification, examples and appended claims are, for convenience, collected here.

I. Definitions

As used herein the term “newly identified MarA responsive gene (NIMR gene)” includes genes newly identified as responding to high constitutive expression or the overexpression of MarA. Preferably, transcription of these genes is directly modulated by MarA, placing them in the mar regulon. As used herein, the term “regulon” includes two or more loci in two or more different operons whose expression is regulated by a common repressor or activator protein. The newly identified mar responsive genes are genes whose expression is controlled by MarA, but which had not, prior to the instant invention, been identified as being under the control of this transcriptional activator and had not been previously identified as part of the mar regulon. NIMR genes can be either positively or negatively regulated by MarA and can respond directly to MarA or can respond indirectly to MarA, e.g., in response to another protein (e.g., a transcriptional regulator) that directly responds to MarA.

NIMR genes do not include genes identified as being part of the “prior art mar regulon.” As used herein, the term “prior art mar regulon” includes: acrAB, fumc, inaA, marA, marB, marR, ompF, ompX, sodA, tolC, and zwf. Preferably, NMIR genes include genes that were not previously associated with stress responses in bacteria. For example, preferred, NIMR genes had not previously been identified as being part of the soxRS regulon (comprising the acna, acrAB, fumC, inaA, mdaA, ompF, ribA, sodA, and zwf genes). Particularly preferred NIMR genes had no known function prior to their placement in the mar regulon in the instant invention. Exemplary NIMR genes are listed in Table 1 below:

TABLE 1

| | |
|---|---|
| accB* (AE000404) | b0357*(AE000142) |
| aceE* (AE000120) | b0447 (AE000151) |
| aceF* (AE000120) | b0853 (AE000187) |
| ackA* (AE000318) | b1448 (AE000241) |
| aldA (AE000239) | b2530*(AE000339) |
| cobU (AE000291) | b2889 (AE000372) |
| fabB* (AE000231) | b2948 (AE000377) |
| fecA* (AE000499) | b3469*(AE000422) |
| galK (AE000178) | mdaB (AE000385) |
| galT (AE000178) | yadG (AE000122) |
| gatA (AE000298) | yadH (AE000122) |
| gatC (AE000298) | ybjC (AE000187) |
| glpD* (AE000418) | yfaE (AE000313) |
| gltA (AE000175) | yggJ (AE000377) |
| gshB (AE000377) | yhbW (AE000397) |
| guaB* (AE000337) | hemB (AE000143) |
| map (AE000126) | mglB (AE000304) |
| mtr (AE000397) | ndh* (AE000211) |
| nfnB (AE000163) | pflB (AE000192) |
| pgi (AE000476) | purA* (AE000195) |
| ribD (AE000148) | rimK (AE000187) |
| rplE* (AE000408) | srlA_2 (AE000354) |
| tnaA (AE000448) | tnaL (AE000448) |
| tpx (AE000230) | ydeA (AE000250) |
| acnA (AE000225) | mdaA (AE000187) |
| ribA (AE000226) | |

Accession numbers from the E. coli K-12 genome project (National Center for Biotechnology Entrez database (http://www.ncbi.nlm.nih.gov/)) are given in parentheses after each gene. The sequences for these exemplary NIMR genes are available on GenBank and are presented in the sequence listing part of the description.
*Indicates a gene that is down regulated by overexpression of MarA.

As used herein, the language “NIMR genes” also includes NIMR genes having nucleotide sequence similarity to the NIMR genes described above. For example, such genes may be derived from other organisms. For instance, the multiple antibiotic resistance (mar) locus, first described in the chromosome of Escherichia coli, is also present among other genera of enteric bacteria (Cohen, S. P., Yan, W. & Levy, S. B. (1993) J. Infect. Dis. 168, 484-488). Molecular characterization of this locus has been performed in E. coli (Cohen, S. P., Hachler, H. & Levy, S. B. (1993) J. Bacteriol. 175, 1484-1492), Salmonella typhimurium (Sulavick, M. C., Dazer, M. & Miller, P. F. (1997) J. Bacteriol. 179, 1857-1866) and more recently Shigella flexneri.

NIMR gene sequences are “structurally related” to one or more of the NIMR genes set forth in the Table above. This structural relatedness can be demonstrated by sequence similarity between two NIMR nucleotide sequences or between the amino acid sequences of two NIMR polypeptides. As used herein, the term “NIMR polypeptide” includes polypeptides specified by NIMR genes. NIMR polypeptides have an NIMR activity, e.g., modulate microbial adaptation to environmental stress and/or microbial virulence.

As used herein, the term “activity” with respect to an NIMR polypeptide includes the modulation of the ability of the microbe to adapt to environmental stress and/or modulation of virulence. In addition, NIMR polypeptides may have additional activities. A used herein, the term “environmental stress” or “environmental challenge” with reference to exposure of a microbe includes agents, which when contacted with a microbe, provoke a stress response in the microbe. Such agents may lead to a decrease in growth, viability, and/or virulence in individual susceptible microbial cells, but also serve as a stimulus for other microbial cells to adapt to the environmental signal e.g., by acting as a selection agent for microbes that have a mutation in a target molecule affected by the stress signal. Thus, in a microbe that is equipped to deal with the environmental stress (e.g., possesses a phenotype that allows growth in response to the changing environmental conditions brought about by the stress signal), the cell adapts, (e.g. retains its virulence and/or its ability to grow and remain viable when exposed to the environmental stress signal). “Environmental stress” or “environmental challenge” refers to agents that come into contact with a microbe or conditions to which a microbe is exposed that present a challenge to the survival of the microbe. Microbes can contact such environmental stress signals inside (including on the surface of) or outside a mammalian body. For example, microbes (e.g., pathogenic microbes) can be contacted with environmental challenges inside the body or microbes outside the body (e.g., pathogenic microbes or environmentally important microbes residing on surfaces) can be contacted with environmental challenges outside the body to create an environmental stress.

In one embodiment an environmental stress or challenge is brought about by human intervention, e.g., by exposure of the microbe to a drug as brought about by man (such as a non-antibiotic agent or an antibiotic). For example, such agents include antibiotics or non-antibiotic compounds.

In another embodiment, an environmental stress or challenge is the result of a natural process, e.g., the natural course of an infection, resulting e.g., in exposure of the microbe to natural anti-infective defenses such as antibodies; exposure of a microbe to increased temperature (e.g., during infection); or exposure of the microbe to an environment lacking in cofactors or vitimins.

As used herein, the term “virulence” includes the degree of pathogenicity of an organism. The term virulence encompasses two features of an organism: its infectivity (the ability to colonize a host) and the severity of the disease produced. As used herein, the term “viability” includes the capacity for cell growth. Viable cells may not actively be multiplying, e.g., may be in a quiescent state, but retain the ability to grow when conditions for growth are more favorable. As used herein, the term “growth” includes the ability to multiply, i.e., by cell division or proliferation.

NIMR polypeptides, before their identification as being regulated by MarA may have been previously found to have one or more other functions, e.g., as set forth in Table 2 below:

TABLE 2

| Physiological function | NIMR genes |
|---|---|
| Energy metabolism, carbon | aceE, aceF, ackA, acnA, |
| | aldA, fumC, glpD, gltA, |
| | mdaA, ndh, pflB, pgi, |
| Biosynthesis of cofactors, carriers | zwf accB, cobU, hemB, |
| | gshB, ribA, ribD |
| Carbon compound catabolism | Galk, galT |
| Amino acid biosynthesis and metabolism | TnaA, tnaL |
| Fatty acid biosynthesis | fabB |
| Nucleotide biosynthesis | GuaB, purA |
| Adaptation | inaA |
| Cell Division | tolC |
| Transport/binding proteins | gatA, gatC, fecA, mglB, mtr, |
| | srlA_2, yadG, yadH, |
| | ydeA, b3469 |
| Protection responses | acrA, marA, marB, marR, |
| | nfnB, sodA, tpx, |
| Cell envelope | OmpF, ompX |
| Ribosome constituents | rimK, rplE |
| Macromolecule synthesis, modification | map |

In isolating or identifying other NIMR molecules, sequence similarity can be shown, e.g., by generating alignments as described in more detail below.

Preferably, NIMR polypeptides share some amino acid sequence identity with a polypeptide encoded by an NIMR gene set forth in the table above. The nucleic acid sequences of the exemplary NIMR genes set forth in the table above and the polypeptides they encode are available in the art. For example, the nucleic acid and amino acid sequences of the exemplary NIMR genes set forth in Table 1 can be found using the accession numbers listed in Table 1 at the NCBI Entrez site (http://www.ncbi.nlm.nih.gov/). These sequences are also presented in Appendix A.

As used herein, the term “nucleic acid molecule(s)” includes polyribonucleotides or polydeoxribonucleotides, which may be unmodified RNA or DNA or modified RNA or DNA. As such, “nucleic acid molecule(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “nucleic acid molecule” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. As used herein, the term “nucleic acid molecule” also includes DNAs or RNAs as described above that contain one or more modified bases. Thus, DNAs or RNAs with backbones modified for stability or for other reasons are “nucleic acid molecule(s)” as that term is intended herein. Moreover, DNAs or RNAs comprising unusual bases, such as inosine, or modified bases, such as tritylated bases, to name just two examples, are nucleic acid molecules as the term is used herein. It will be appreciated that a great variety of modifications have been made to DNA and RNA that serve many useful purposes known to those of skill in the art. The term “nucleic acid molecule(s)” as it is employed herein embraces such chemically, enzymatically or metabolically modified forms of nucleic acid molecules, as well as the chemical forms of DNA and RNA characteristic of viruses and cells, including, for example, simple and complex cells. “Nucleic acid molecule(s)” also embraces short nucleic acid molecules often referred to as oligonucleotide(s).

Preferred NIMR nucleic acid molecules are isolated. An “isolated” nucleic acid molecule is one that is separated from other nucleic acid molecules which are present in the natural source of the nucleic acid. For example, with regard to genomic DNA, (e.g. whether chromosomal or episomal) the term “isolated” includes nucleic acid molecules which are separated from flanking DNA sequences with which the DNA is naturally associated. Preferably, an “isolated” nucleic acid molecule is free of sequences which naturally flank the nucleic acid molecule (i.e., sequences located at the 5′ and 3′ ends of the nucleic acid molecule) in the DNA (e.g., chromosomal or episomal) of the organism from which the nucleic acid molecule is derived. As such, isolated DNA is not in its naturally occurring state (although, as described in more detail below, its sequence may be naturally occurring in the sense that has not been altered (e.g., mutated) from its naturally occurring form). For example, in various embodiments, an isolated NIMR nucleic acid molecule can contain less than about 5 kb, 4 kb, 3 kb, 2 kb, 1 kb, 0.5 kb, 0.1 kb, or 0.05 kb of nucleotide sequences which naturally flank the nucleic acid molecule in DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. An “isolated” NIMR nucleic acid molecule may, however, be linked to other nucleotide sequences that do not normally flank the NIMR sequences in genomic DNA (e.g., the NIMR nucleotide sequences may be linked to vector sequences). In certain preferred embodiments, an “isolated” nucleic acid molecule, such as a cDNA molecule, also may be free of other cellular material. However, it is not necessary for the NIMR nucleic acid molecule to be free of other cellular material to be considered “isolated” (e.g., an NIMR DNA molecule separated from other chromosomal DNA and inserted into another bacterial cell would still be considered to be “isolated”).

As used herein, “polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2^{nd} Ed., T. E. Creighton, W. H. Freeman and Company, New York (1993) and Wold, F., Posttranslational Protein Modifications: Perspectives and Prospects, pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

As used herein, an “isolated polypeptide” or “isolated protein” refers to a polypeptide or protein that is substantially free of other polypeptides, proteins, cellular material and culture medium when isolated from cells or produced by recombinant DNA techniques, or chemical precursors or other chemicals when chemically synthesized. An “isolated” or “purified” polypeptide or biologically active portion thereof is substantially free of cellular material or other contaminating polypeptides from the cell or tissue source from which the NIMR polypeptide is derived, or substantially free from chemical precursors or other chemicals when chemically synthesized. The language “substantially free of cellular material” includes preparations of NIMR polypeptide in which the polypeptide is separated from cellular components of the cells from which it is isolated or recombinantly produced. In one embodiment, the language “substantially free of cellular material” includes preparations of NIMR polypeptide having less than about 30% (by dry weight) of non-NIMR polypeptide (also referred to herein as a “contaminating polypeptide”), more preferably less than about 20% of non-NIMR polypeptide, still more preferably less than about 10% of non-NIMR polypeptide, and most preferably less than about 5% non-NIMR polypeptide. When the NIMR polypeptide or biologically active portion thereof is recombinantly produced, it is also preferably substantially free of culture medium, i.e., culture medium represents less than about 20%, more preferably less than about 10%, and most preferably less than about 5% of the volume of the polypeptide preparation.

The language “substantially free of chemical precursors or other chemicals” includes preparations of NIMR polypeptide in which the polypeptide is separated from chemical precursors or other chemicals that are involved in the synthesis of the polypeptide. In one embodiment, the language “substantially free of chemical precursors or other chemicals” includes preparations of NIMR polypeptide having less than about 30% (by dry weight) of chemical precursors or non-NIMR chemicals, more preferably less than about 20% chemical precursors or non-NIMR chemicals, still more preferably less than about 10% chemical precursors or non-NIMR chemicals, and most preferably less than about 5% chemical precursors or non-NIMR chemicals.

Preferred NIMR nucleic acid molecules and polypeptides are “naturally occurring.” As used herein, a “naturally-occurring” molecule refers to an NIMR molecule having a nucleotide sequence that occurs in nature (e.g., encodes a natural NIMR polypeptide). In addition, naturally or non-naturally occurring variants of these polypeptides and nucleic acid molecules which retain the same functional activity, e.g., the ability to modulate adaptation to stress and/or virulence in a microbe. Such variants can be made, e.g., by mutation using techniques that are known in the art. Alternatively, variants can be chemically synthesized.

As used herein the term “variant(s)” includes nucleic acid molecules or polypeptides that differ in sequence from a reference nucleic acid molecule or polypeptide, but retain its essential properties. Changes in the nucleotide sequence of the variant may or may not alter the amino acid sequence of a polypeptide encoded by the reference nucleic acid molecule. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that-the sequences of the reference polypeptide and the variant are closely similar overall and, in many regions, identical. A variant and reference polypeptide may differ in amino acid sequence by one or more substitutions, additions, and/or deletions in any combination. A variant of a nucleic acid molecule or polypeptide may be naturally occurring, such as an allelic variant, or it may be a variant that is not known to occur naturally. Non-naturally occurring variants of nucleic acid molecules and polypeptides may be made by mutagenesis techniques, by direct synthesis, and by other recombinant methods known to skilled artisans.

For example, it will be understood that the NIMR polypeptides described herein are also meant to include equivalents thereof. Such variants can be made, e.g., by mutation using techniques that are known in the art. Alternatively, variants can be chemically synthesized. For instance, mutant forms of NIMR polypeptides which are functionally equivalent, (e.g., have the ability to bind to DNA and to regulate transcription from an operon) can be made using techniques which are well known in the art. Mutations can include, e.g., at least one of a discrete point mutation which can give rise to a substitution, or by at least one deletion or insertion. For example, random mutagenesis can be used. Mutations can also be made by random mutagenesis or using cassette mutagenesis. For the former, the entire coding region of a molecule is mutagenized by one of several methods (chemical, PCR, doped oligonucleotide synthesis) and that collection of randomly mutated molecules is subjected to selection or screening procedures. In the latter, discrete regions of a polypeptide, corresponding either to defined structural or functional determinants are subjected to saturating or semi-random mutagenesis and these mutagenized cassettes are re-introduced into the context of the otherwise wild type allele. In one embodiment, PCR mutagenesis can be used. For example, Megaprimer PCR can be used (O. H. Landt, 1990. Gene 96:125-128).

In certain embodiments, such variants have at least about 25, 30, 35, 40, 45, 50, or 60% or more amino acid identity with a naturally occurring NIMR polypeptide. In preferred embodiments, such variants have at least about 70% amino acid identity with a naturally occurring NIMR polypeptide. In more preferred embodiments, such variants have at least about 80% amino acid identity with a naturally occurring NIMR polypeptide. In particularly preferred embodiments, such variants have at least about 90% amino acid identity and preferably at least about 95% amino acid identity with a naturally occurring NIMR polypeptide.

In yet other embodiments, a nucleic acid molecule encoding a variant of an NIMR polypeptide is capable of hybridizing under stringent conditions to a nucleic molecule encoding a naturally occurring NIMR polypeptide.

Preferred NIMR nucleic acid molecules and NIMR polypeptides are “naturally occurring.” As used herein, a “naturally-occurring” molecule refers to an NIMR polypeptide encoded by a nucleotide sequence that occurs in nature (e.g., encodes a natural NIMR polypeptide). Such molecules can be obtained from other microbes, e.g., based on their sequence similarity to the NIMR molecules described herein.

In addition, naturally or non-naturally occurring variants of these polypeptides and nucleic acid molecules which retain the same functional activity, e.g., the ability to modulate microbial responses to environmental stress and, thereby, modulate microbial adaptation to stress and/or microbial virulence are also within the scope of the invention. Such variants can be made, e.g., by mutation using techniques which are known in the art. Alternatively, variants can be chemically synthesized.

As used herein, “heterologous DNA” or “heterologous nucleic acid” includes DNA that does not occur naturally in the cell (e.g., as part of the genome) in which it is present or which is found in a location or locations in the genome that differs from that in which it occurs in nature or which is operatively linked to DNA to which it is not normally linked in nature (i.e., a gene that has been operatively linked to a heterologous promoter). Heterologous DNA is 1) not naturally occurring in a particular position (e.g., at a particular position in the genomp) or 2) is not endogenous to the cell into which it is introduced, but has been obtained from another cell. Heterologous DNA can be from the same species or from a different species. Any DNA that one of skill in the art would recognize or consider as heterologous or foreign to the cell in which is expressed is herein encompassed by the term heterologous DNA.

The terms “heterologous protein”, “recombinant protein”, and “exogenous protein” are used interchangeably throughout the specification and refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid molecule.

The term “interact” includes close contact between molecules that results in a measurable effect, e.g., on the conformation and/or activity of at least one of the molecules involved in the interaction. For example, a first molecule can be said to interact with a second when it inhibits the binding of the second molecule to a target (e.g., a DNA or polypeptide target) to which that second molecule normally binds, or when it alters the activity of the second molecule, e.g., by steric interaction with a domain of the second molecule that mediates its activity. For example, compounds can interact with (e.g., by binding) to an NIMR polypeptide and alter the activity of the NIMR polypeptide or can interact with (e.g., by binding) to an NIMR nucleic acid molecule and alter transcription of an NIMR polypeptide from that nucleic acid molecule.

As used herein, the term “NIMR binding polypeptide” includes polypeptides that normally interact with NIMR nucleic acid molecules or NIMR polypeptides under physiological conditions in a cell, e.g., and alter transcription of an NIMR nucleic acid molecule or activity of an NIMR polypeptide.

As used herein, the term “drug” includes antibiotic agents and non-antibiotic agents. The term “drug” includes antiinfective compounds which are static or cidal for microbes, e.g., an antimicrobial compound which inhibits the growth and/or viability of a microbe. Preferred antiinfective compounds increase the susceptibility of microbes to antibiotics or decrease the infectivity or virulence of a microbe. The term “drug” includes the antimicrobial agents such as disinfectants, antiseptics, and surface delivered compounds. For example, antibiotics or other types of antibacterial compounds, including agents which induce oxidative stress, and organic solvents are included in this term. The term “drug” also includes biocides. The term “biocide” is art recognized and includes an agent that is thought to kill a cell “non-specifically,” or a broad spectrum agent whose mechanism of action is unknown as well as drugs that are known to be target-specific (e.g., triclosan). Examples of biocides include paraben, chlorbutanol, phenol, alkylating agents such as ethylene oxide and formaldehyde, halides, mercurials and other heavy metals, detergents, acids, alkalis, and chlorhexidine. Other biocidal agents include: pine oil, quaternary amine compounds such as alkyl dimethyl benzyl ammonium chloride, chloroxylol, chlorhexidine, cyclohexidine, triclocarbon, and disinfectants. The term “bactericidal” refers to an agent that can kill a bacterium; “bacteriostatic” refers to an agent that inhibits the growth of a bacterium.

The term “antibiotic” is art recognized and includes antimicrobial agents synthesized by an organism in nature and isolated from this natural source, and chemically synthesized drugs. The term includes but is not limited to: polyether ionophores such as monensin and nigericin; macrolide antibiotics such as erythromycin and tylosin; aminoglycoside antibiotics such as streptomycin and kanamycin; β-lactam antibiotics (having a β lactam ring) such as penicillin and cephalosporin; and polypeptide antibiotics such as subtilisin and neosporin. Semi-synthetic derivatives of antibiotics, and antibiotics produced by chemical methods are also encompassed by this term. Chemically-derived antimicrobial agents such as isoniazid, trimethoprim, quinolones, fluoroquinolones and sulfa drugs are considered antibacterial drugs, and the term antibiotic includes these. It is within the scope of the screens of the present invention to include compounds derived from natural products and compounds that are chemically synthesized.

The phrase “non-antibiotic agent” includes agents that are not art recognized as being antibiotics. Exemplary non-antibiotic agents include, e.g., biocides, disinfectants or antiinfectives. Non antibiotic agents also include compounds incorporated into consumer goods, e.g., for topical use on a subject or as cleaning products. In contrast to the term “biocide,” an antibiotic or an “anti-microbial drug approved for human use” is considered to have a specific molecular target in a microbial cell. Preferably a microbial target of a therapeutic agent is sufficiently different from its physiological counterpart in a subject in need of treatment that the antibiotic or drug has minimal adverse effects on the subject.

The term “microbe” includes microorganisms expressing or made to express an NMIR polypeptide. “Microbes” are of some economic importance, e.g., are environmentally inportant or are important as human pathogens. For example, in one embodiment microbes cause environmental problems, e.g., fouling or spoilage, or perform useful functions such as breakdown of plant matter. In another embodiment, microbes are organisms that live in or on mammals and are medically important. Preferably microbes are unicellular and include bacteria, fungi, or protozoa. In another embodiment, microbes suitable for use in the invention are multicellular, e.g., parasites or fungi. In preferred embodiments, microbes are pathogenic for humans, animals, or plants. Microbes may be used as intact cells or as sources of materials for cell-free assays as described herein.

As used herein the term “reporter gene” includes any gene that encodes an easily detectable product that is operably linked to a promoter. By operably linked it is meant that under appropriate conditions an RNA polymerase may bind to the promoter of the regulatory region and proceed to transcribe the nucleotide sequence of the reporter gene. In certain embodiments, however, it may be desirable to include other sequences, e.g., transcriptional regulatory sequences, in the reporter gene construct. For example, modulation of the activity of the promoter may be affected by altering the RNA polymerase binding to the promoter region, or, alternatively, by interfering with initiation of transcription or elongation of the mRNA. Thus, sequences which are herein collectively referred to as transcriptional regulatory elements or sequences may also be included in the reporter gene construct. In addition, the construct may include sequences of nucleotides that alter translation of the resulting mRNA, thereby altering the amount of reporter gene product.

As used herein the term “test compound” includes agent(s) that are tested using the assays of the invention to determine whether they modulate the activity or expression of an NIMR polypeptide. More than one compound, e.g., a plurality of compounds, can be tested at the same time for their ability to modulate the activity or expression of an NIMR polypeptide sequence in a screening assay.

Test compounds that can be assayed in the subject assays include antibiotic and non-antibiotic compounds. In one embodiment, test compounds include candidate detergent or disinfectant compounds. Exemplary compounds which can be screened for activity include, but are not limited to, peptides, non-peptidic compounds, nucleic acids, carbohydrates, small organic molecules (e.g., polyketides), and natural product extract libraries. The term “non-peptidic compound” is intended to encompass compounds that are comprised, at least in part, of molecular structures different from naturally-occurring L-amino acid residues linked by natural peptide bonds. However, “non-peptidic compounds” are intended to include compounds composed, in whole or in part, of peptidomimetic structures, such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds. “Non-peptidic compounds” also are intended to include natural products.

As used herein, the term “antibody” is intended to include immunoglobulin molecules and immunologically active portions of immunoglobulin molecules, i.e., molecules that contain an antigen binding site which binds (immunoreacts with) an antigen, such as Fab and F(ab′)₂ fragments, single chain antibodies, intracellular antibodies, scFv, Fd, or other fragments. Preferably, antibodies of the invention bind specifically or substantially specifically to NIMR molecules. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition thus typically display a single binding affinity for a particular antigen with which it immunoreacts.

The phrase “specifically” with reference to binding, recognition, or reactivity of antibodies includes antibodies which bind to a naturally occurring NIMR molecule, but are substantially unreactive with other unrelated molecules. Preferably, such antibodies bind to an NIMR molecule (or its homolog from another species) and bind to non-NIMR molecules (or unrelated NIMR molecules) with only background binding. Antibodies specific for NIMR family molecules from one source may or may not be reactive with NIMR molecules from different species. Antibodies specific for naturally occurring NIMR molecules may or may not bind to mutant forms of such molecules. Assays to determine affinity and specificity of binding are known in the art, including competitive and non-competitive assays. Assays of interest include ELISA, RIA, flow cytometry, etc.

II. Compositions Which Modulate Antibiotic Resistance

A. Nucleic Acid Molecules

In one aspect, the invention provides isolated nucleic acid molecules comprising or consisting essentially NIMR nucleotide sequences. In another aspect, the invention provides nucleic acid molecules consisting of NIMR nucleotide sequences. Exemplary NIMR molecules are shown in Table 1.

NIMR genes have structural similarity (e.g., to the sequence shown in Table 1) and, preferably, encode NIMR polypeptides having an NIMR polypeptide activity. For example, in one embodiment, an NIMR polypeptide is capable of modulating microbial responses to environmental stress and, thereby, modulating microbial adaptation to stress and/or microbial virulence. Preferably, NIMR polypeptidess modulate resistance to drugs. In one embodiment, NIMR polypeptides modulate resistance to non-antibiotic compounds. In another embodiment, NIMR polypeptidess modulate resistance to antibiotics.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid molecule and the amino acid sequence encoded by that nucleic acid molecule, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid | GAC, GAT |
| (Asp, D) | |
| Cysteine (Cys, C) | TGC, TGT |
| Glutamic acid | GAA, GAG |
| (Glu, E) | |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine | TTC, TTT |
| (Phe, F) | |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA molecule coding for an NIMR polypeptide of the invention (or a portion thereof) can be used to derive the NIMR amino acid sequence, using the genetic code to translate the DNA or RNA molecule into an amino acid sequence. Likewise, for any NIMR-amino acid sequence, corresponding nucleotide sequences that can encode an NIMR protein can be deduced from the genetic code (which, because of its redundancy, will produce multiple nucleic acid sequences for any given amino acid sequence). Thus, description and/or disclosure herein of an NIMR related nucleotide sequence should be considered to also include description and/or disclosure of the amino acid sequence encoded by the nucleotide sequence. Similarly, description and/or disclosure of an NIMR amino acid sequence herein should be considered to also include description and/or disclosure of all possible nucleotide sequences that can encode the amino acid sequence.

One aspect of the invention pertains to isolated nucleic acid molecules that encode NIMR proteins or biologically active portions thereof, as well as nucleic acid fragments sufficient for use as hybridization probes to identify NIMR-encoding nucleic acids (e.g., NIMR mRNA) and fragments for use as PCR primers for the amplification or mutation of NIMR nucleic acid molecules. It will be understood that in discussing the uses of NIMR nucleic acid molecules, e.g., as shown in Table 1, that fragments of such nucleic acid molecules as well as full length NIMR nucleic acid molecules can be used.

A nucleic acid molecule of the present invention, e.g., a nucleic acid molecule having the nucleotide sequence of an NIMR molecule shown in Table 1, or a portion thereof, can be isolated using standard molecular biology techniques and the sequence information provided herein. For example, using all or portion of an NIMR nucleic acid sequence as a hybridization probe, NIMR nucleic acid molecules can be isolated using standard hybridization and cloning techniques (e.g., as described in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989). Moreover, a nucleic acid molecule encompassing all or a portion of an NIMR nucleotide sequence can be isolated by the polymerase chain reaction (PCR) using synthetic oligonucleotide primers designed based upon an NIMR nucleotide sequence (e.g., from a different species of microbe).

A nucleic acid molecule of the invention can be amplified using cDNA, mRNA or alternatively, genomic DNA, as a template and appropriate oligonucleotide primers according to standard PCR and/or RT PCR amplification techniques. The nucleic acid so amplified can be cloned into an appropriate vector and characterized by DNA sequence analysis. Furthermore, oligonucleotides corresponding to NIMR nucleotide sequences can be prepared by standard synthetic techniques, e.g., using an automated DNA synthesizer.

In another preferred embodiment, an isolated nucleic acid molecule of the invention comprises a nucleic acid molecule which is a complement of a nucleotide sequence of an NIMR gene presented in Table 1 or a portion of the nucleotide sequence. A nucleic acid molecule which is complementary to the nucleotide sequence of an NIMR gene shown in Table 1 is one which is sufficiently complementary to the nucleotide sequence of an NIMR gene presented in Table 1, such that it can hybridize to the nucleotide sequence of an NIMR gene shown in Table 1, thereby forming a stable duplex.

In addition to the nucleic acid molecule shown in Table 1, other NIMR nucleotide sequences of the invention are “structurally related” (i.e., share sequence identity with) the NIMR nucleotide sequence of the NIMR molecules listed in Table 1. Such sequence similarity can be shown, e.g., by optimally aligning the NIMR nucleotide sequence with a putative NIMR nucleotide sequence using an alignment program for purposes of comparison and comparing corresponding positions. In a preferred embodiment, an isolated nucleic acid molecule of the invention comprises the nucleotide sequence of one of the molecules listed in Table 1.

In still another preferred embodiment, an isolated nucleic acid molecule of the present invention comprises a nucleotide sequence which is at least about 25, 30, 35, 40, 45, 50, or 60% or more homologous to a naturally occurring NIMR polypeptide. In another embodiment, an isolated nucleic acid molecule of the invention comprises a nucleotide sequence which is at least about 25, 30, 35, 40, 45, 50, or 60% or more amino acid identity with a naturally occurring NIMR polypeptide. In another embodiment, an isolated nucleic acid molecule of the invention comprises a nucleotide sequence which is at least about 65%, 70%, 75%, 80%, 85%, 90%, 95%, 98% or more homologous to a nucleotide sequence (e.g., to the entire length of a nucleotide sequence) of an NIMR molecule shown in Table 1 or a portion thereof.

In other embodiments, a nucleic acid molecule of the invention has at least 25, 30, 35, 40, 45, 50, 60, or 70% identity, more preferably 80% identity, and even more preferably 90% identity with a nucleic acid molecule comprising: at least about 100, 200, 300, 400, 500, 600, or at about 700 nucleotides of an NIMR molecule listed in Table 1.

Sequence similarity can be shown, e.g., by optimally aligning NIMR nucleotide or amino acid sequences for purposes of comparison using an alignment program and comparing corresponding positions of the sequences. To determine the degree of similarity between sequences, they can be aligned for optimal comparison purposes (e.g., gaps may be introduced in the sequence of one polypeptide or nucleic acid molecule for optimal alignment with the other polypeptide or nucleic acid molecule with which they are to be compared). The amino acid residues or bases at a given position are then compared with the corresponding amino acid residue or base in the sequence with which they are being compared. When a position in one sequence is occupied by the same amino acid residue or by the same base as the corresponding position in the other sequence, then the sequences are identical at that position. If amino acid residues are not identical, they may be similar. As used herein, an amino acid residue is “similar” to another amino acid residue if the two amino acid residues are members of the same family of residues having similar side chains. Families of amino acid residues having similar side chains have been defined in the art (see, for example, Altschul et al. 1990. J. Mol. Biol. 215:403) including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan.) The degree (percentage) of similarity between sequences, therefore, is a function of the number of identical or similar positions shared by two sequences (i.e., % homology=# of identical or similar positions/total # of positions×100). Alignment strategies are well known in the art; see, for example, Altschul et al. supra for optimal sequence alignment.

Nucleic acid molecules that exist as an active functional unit, e.g., mRNA molecules, will be expected to have a higher degree of structural identity among homologs. It will be understood that among divergent organisms, there will be a lower degree of structural relatedness among the nucleic acid molecules that encode functional homologs.

Preferably, NIMR polypeptides share some amino acid sequence similarity with a polypeptide encoded by an NIMR gene of a molecule listed in Table 1. The nucleic acid and/or amino acid sequences of an NIMR gene or polypeptide (e.g., as provided above) can be used as “query sequence” to perform a search against databases (e.g., either public or private such as http://www.tigr.org) to, for example, identify other NIMR genes (or polypeptides) having related sequences. For example, such searches can be performed, e.g., using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the above NIMR nucleic acid molecules. BLAST polypeptide searches can be performed with the XBLAST program, score=50, wordlength=3 to obtain amino acid sequences homologous to NIMR polypeptide molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

However, it will be understood that the level of sequence identity among microbial genes, even though members of the same family, is not necessarily high. This is particularly true in the case of divergent genomes where the level of sequence identity may be low, e.g., less than 20% (e.g., B. burgdorferi as compared e.g., to B. subtilis). Accordingly, structural similarity among NIMR-molecules can also be determined based on “three-dimensional correspondence” of amino acid residues. As used herein, the language “three-dimensional correspondence” is meant to includes residues which spatially correspond, e.g., are in the same functional position of an NIMR polypeptide member as determined, e.g., by x-ray crystallography, but which may not correspond when aligned using a linear alignment program. The language “three-dimensional correspondence” also includes residues which perform the same function, e.g., bind to DNA or bind the same cofactor, as determined, e.g., by mutational analysis.

Nucleic acid molecules that differ in nucleotide sequence from those NIMR molecules listed in Table 1 due to degeneracy of the genetic code, and thus encode the same NIMR protein as are encompassed by the invention. Accordingly, in another embodiment, an isolated nucleic acid molecule of the invention has a nucleotide sequence encoding a protein having an amino acid sequence of an NIMR molecule listed in Table 1.

In addition to the nucleotide sequences of the NIMR molecules shown in Table 1, it will be appreciated by those skilled in the art that DNA sequence polymorphisms that lead to changes in the amino acid sequences of a given NIMR polypeptide may exist within a population of organisms. Such nucleotide variations and resulting amino acid polymorphisms in NIMR genes that are the result of natural allelic variation and that do not alter the functional activity of an NIMR polypeptide are intended to be within the scope of the invention.

Moreover, nucleic acid molecules encoding functional NIMR polypeptides but which have a nucleotide sequence which differs from an NIMR nucleotide sequence of a molecule listed in Table 1 are intended to be within the scope of the invention. Nucleic acid molecules encoding functional homologs of the NIMR proteins listed in Table 1, e.g., from different species, and thus which have a nucleotide sequence which differs from the NIMR sequence of the NIMR molecules listed in Table 1 are intended to be within the scope of the invention. Given the list of NIMR genes set forth in Table 1, NIMR homologs can be readily identified by one of ordinary skill in the art, e.g., by structural similarity to the NIMR nucleotide sequences provided using standard techniques.

For example, NIMR nucleic acid molecules can be identified as being structurally similar to the exemplary NIMR gene set forth herein based on their ability to hybridize to the nucleic acid molecule listed in Table 1 under stringent conditions. For example, an NIMR DNA can be isolated from a DNA library using all or portion of a nucleotide sequence of an NIMR molecule from Table 1 as a hybridization probe and standard hybridization techniques (e.g., as described in Sambrook, J., et al. Molecular Cloning: A Laboratory Manual. 2nd, ed., Cold Spring Harbor Laboratory, Cold Spring Harbor, N.Y., 1989; Cohen et al. 1993. J. of Infectious Diseases. 168:484)).

As used herein, the term “hybridizes under stringent conditions” is intended to describe conditions for hybridization and washing under which nucleotide sequences at least 30%, 40%, 50%, or 60% homologous to each other typically remain hybridized to each other. Preferably, the conditions are such that sequences at least about 70%, more preferably at least about 80%, even more preferably at least about 85% or 90% homologous to each other typically remain hybridized to each other. Preferably, an isolated nucleic acid molecule of the invention that hybridizes under stringent conditions to the-sequence of a molecule from Table 1 or its complement corresponds to a naturally-occurring nucleic acid molecule. Such stringent conditions are known to those skilled in the art and can be found e.g., in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Conditions for hybridizations are largely dependent on the melting temperature Tm that is observed for half of the molecules of a substantially pure population of a double-stranded nucleic acid. Tm is the temperature in ° C. at which half the molecules of a given sequence are melted or single-stranded. For nucleic acids of sequence 11 to 23 bases, the Tm can be estimated in degrees C. as 2 (number of A+T residues)+4(number of C+G residues). Hybridization or annealing of nucleic acid molecules should be conducted at a temperature lower than the Tm, e.g., 15° C., 20° C., 25° C. or 30° C. lower than the Tm. The effect of salt concentration (in M of NaCl) can also be calculated, see for example, Brown, A., “Hybridization” pp. 503-506, in The Encyclopedia of Molec. Biol., J. Kendrew, Ed., Blackwell, Oxford (1994).

In addition, NIMR genes can be identified by overexpressing transcriptional activators related to MarA in other microbes and identifying the genes whose expression is controlled by overexpression of the MarA homolog, using techniques similar to those set forth in the instant examples.

Moreover, the nucleic acid molecules of the invention can comprise only a portion of a full length NIMR nucleic acid sequence. For example a fragment can be used as a probe or primer or a fragment encoding a biologically active portion of an NIMR protein. The nucleotide sequence of the NIMR genes allows for the generation of probes and primers designed for use in identifying and/or cloning other NIMR polypeptides, as well as NIMR homologues from other species. The probe/primer typically comprises a substantially purified oligonucleotide. In one embodiment, the oligonucleotide comprises a region of nucleotide sequence that hybridizes under stringent conditions to at least about 12 or 15, preferably about 20 or 25, more preferably about 30, 35, 40, 45, 50, 55, 60, 65, 75, or 100 consecutive nucleotides of a sense sequence of an NIMR molecule from Table 1 or of a naturally occurring allelic variant or mutant thereof. In another embodiment, a nucleic acid molecule of the present invention comprises a nucleotide sequence which is at least about 200, 300, 400, 500, 600 or 700 nucleotides in length and hybridizes under stringent hybridization conditions to a nucleic acid molecule of Table 1 or the complement thereof.

Moreover, a nucleic acid molecule encompassing all or a portion of an NIMR gene can be isolated by the polymerase chain reaction using oligonucleotide primers designed based upon the sequence of an NIMR molecule listed in Table 1. For example, RNA can be isolated from cells (e.g., by the guanidinium-thiocyanate extraction procedure of Chirgwin et al. (1979) Biochemistry 18: 5294-5299). and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon an NIMR nucleotide sequence. A nucleic acid molecule of the invention can be amplified using cDNA or, alternatively, genomic DNA, as a template and appropriate oligonucleotide primers according to standard PCR amplification techniques. The nucleic acid so amplified can be sequenced directly or cloned into an appropriate vector and characterized by DNA sequence analysis. Furthermore, oligonucleotides corresponding to an NIMR nucleotide sequence can be prepared by standard synthetic techniques, e.g., using an automated DNA synthesizer.

In addition to naturally-occurring allelic variants of NIMR sequences that may exist in a population, the skilled artisan will further appreciate that minor changes may be introduced by mutation into an NIMR nucleotide sequences, e.g., of a molecule listed in Table 1, thereby leading to changes in the amino acid sequence of the encoded polypeptide, without altering the functional activity of an NIMR polypeptide. For example, nucleotide substitutions leading to amino acid substitutions at “non-essential” amino acid residues may be made in the sequence of an NIMR molecule of Table 1. A “non-essential” amino acid residue is a residue that can be altered from the wild-type sequence of an NIMR nucleic acid molecule (e.g., the sequence of an NIMR molecule listed in Table 1) without altering the functional activity of an NIMR molecule. Exemplary residues which are non-essential and, therefore, amenable to substitution, can be identified by one of ordinary skill in the art, e.g., by performing an amino acid alignment of NIMR molecules (e.g., NIMR homologs from different species) and determining residues that are not conserved or by alanine scanning mutagenesis. Such residues, because they have not been conserved, are more likely amenable to substitution.

Accordingly, another aspect of the invention pertains to nucleic acid molecules encoding NIMR proteins that contain changes in amino acid residues that are not essential for an NIMR activity. Such NIMR proteins differ in amino acid sequence from an NIMR molecule listed in Table 1, yet retain an inherent NIMR activity. An isolated nucleic acid molecule encoding a non-natural variant of an NIMR polypeptide can be created by introducing one or more nucleotide substitutions, additions or deletions into the nucleotide sequence of an NIMR molecule of Table 1 such that one or more amino acid substitutions, additions or deletions are introduced into the encoded polypeptide. Mutations can be introduced into an NIMR molecule by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an NIMR polypeptide is preferably replaced with another amino acid residue from the same side chain family.

Alternatively, in another embodiment, mutations can be introduced randomly along all or part of an NIMR coding sequence, such as by saturation mutagenesis, and the resultant mutants can be screened for activity, to identify mutants that retain functional activity. Following mutagenesis, the encoded NIMR mutant polypeptide can be expressed recombinantly in a host cell and the functional activity of the mutant polypeptide can be determined using assays available in the art for assessing an NIMR activity.

Yet another aspect of the invention pertains to isolated nucleic acid molecules encoding an NIMR fusion polypeptide. Such nucleic acid molecules, comprising at least a first nucleotide sequence encoding a full-length (an entire) NIMR protein, polypeptide or peptide having an NIMR activity operatively linked to a second nucleotide sequence encoding a non-NIMR protein, polypeptide or peptide, can be prepared by standard recombinant DNA techniques.

In addition to the nucleic acid molecules encoding NIMR proteins described above, another aspect of the invention pertains to isolated nucleic acid molecules which are antisense thereto. An “antisense” nucleic acid comprises a nucleotide sequence which is complementary to a “sense” nucleic acid encoding a polypeptide, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire NIMR coding strand, or only to a portion thereof. In one embodiment, an antisense nucleic acid molecule is antisense to a “coding region” of the coding strand of a nucleotide sequence encoding NIMR. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding NIMR. The term “noncoding region” refers to 5′ and 3′ sequences which flank the coding region that are not translated into amino acids.

With the coding strand sequences encoding NIMR molecules disclosed herein, antisense nucleic acids of the invention can be designed according to the rules of Watson and Crick base pairing. The antisense nucleic acid molecule can be complementary to the entire coding region of NIMR mRNA, but more preferably is an oligonucleotide which is antisense to only a portion of the coding or noncoding region of NIMR mRNA. For example, the antisense oligonucleotide can be complementary to the region surrounding the translation start site of NIMR mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules of the invention are typically administered to a subject or generated in situ such that they hybridize with or bind to cellular nucleic acid molecules to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave NIMR mRNA transcripts to thereby inhibit translation of NIMR mRNA. A ribozyme having specificity for an NIMR-encoding nucleic acid can be designed based upon the nucleotide sequence of SEQ ID NO:1. For example, a derivative of a Tetrahymena L-19 IVS RNA can be constructed in which the nucleotide sequence of the active site is complementary to the nucleotide sequence to be cleaved in an NIMR-encoding mRNA. See, e.g., Cech et al. U.S. Pat. No. 4,987,071; and Cech et al. U.S. Pat. No. 5,116,742. Alternatively, NIMR mRNA can be used to select a catalytic RNA having a specific ribonuclease activity from a pool of RNA molecules. See, e.g., Bartel, D. and Szostak, J. W. (1993) Science 261:1411-1418.

Alternatively, gene expression can be inhibited by targeting nucleotide sequences complementary to the regulatory region of NIMR (e.g., the NIMR promoter and/or enhancers) to form triple helical structures that prevent transcription of the NIMR gene in target cells. See generally, Helene, C. (1991) Anticancer Drug Des. 6(Alekshun, M. A. & Levy, S. B. (1999) J. Bacteriol. 181, 4669-4672):569-84; Helene, C. et al. (1992) Ann. N.Y. Acad. Sci. 660:27-36; and Maher, L. J. (1992) Bioassays 14(12):807-15.

In yet another embodiment, the NIMR nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (George, A. M. & Levy, S. B. (1983) J. Bacteriol. 155, 541-548): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of NIMR nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of NIMR nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of NIMR molecules can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of NIMR nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (Hamilton, C. M., Aldea, M., Washburn, B. K., Babitzke, P. & Kushner, S. R. (1989) J. Bacteriol. 171, 4617-4622): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

B. NIMR Polypeptides, Fragments thereof, and Anti-NIMR Antibodies

One aspect of the invention pertains to isolated NIMR polypeptides, and biologically active portions thereof, as well as polypeptide fragments suitable for use as immunogens to raise anti-NIMR antibodies.

In one embodiment, native NIMR polypeptides can be isolated from cells or tissue sources by an appropriate purification scheme using standard polypeptide purification techniques. In another embodiment, NIMR polypeptides are produced by recombinant DNA techniques. Alternative to recombinant expression, an NIMR polypeptide or polypeptide can be synthesized chemically using standard peptide synthesis techniques. It will be understood that in discussing the uses of NIMR polypeptides, e.g., as shown in Table 1, that fragments of such polypeptides that are not full length NIMR polypeptides as well as full length NIMR polypeptides can be used.

Preferably, the NIMR polypeptides comprise the amino acid sequence encoded by the nucleotide sequence of an NIMR molecule or a portion thereof. In another preferred embodiment, the polypeptide comprises the amino acid sequence of an NIMR molecule listed in Table 1 or a portion thereof.

Preferred NIMR polypeptides are naturally occurring. In other embodiments, the polypeptide has at least about 25, 30, 35, 40, 45, 50, or 60% or more amino acid identity with a naturally occurring NIMR polypeptide. Preferably, the polypeptide has at least about 70% amino acid identity, more preferably 80%, and even more preferably, 90% or 95% amino acid identity with the amino acid sequence of an NIMR molecule shown in Table 1 or a portion thereof. Preferred portions of NIMR polypeptide molecules are biologically active, i.e., encode a portion of the NIMR polypeptide having the ability to modulate microbial responses to environmental stress and, thereby, modulate microbial adaptation to stress and/or microbial virulence.

In addition, naturally or non-naturally occurring variants of these polypeptides and nucleic acid molecules which retain the same functional activity, e.g., the ability to modulate drug resistance in a cell are also within the scope of the invention. Such variants can be made, e.g., by mutation using techniques which are known in the art. Alternatively, variants can be chemically synthesized.

For example, it will be understood that the NIMR polypeptides described herein also encompass equivalents thereof. For instance, mutant forms of NIMR polypeptides which are functionally equivalent, (e.g., modulate resistance to environmental challenge) can be made using techniques which are well known in the art. Mutations can include, e.g., at least one of a discrete point mutation which can give rise to a substitution, or by at least one deletion or insertion. For example, random mutagenesis can be used. Mutations can be made by random mutagenesis or using cassette mutagenesis. For the former, the entire coding region of a molecule is mutagenized by one of several methods (chemical, PCR, doped oligonucleotide synthesis) and that collection of randomly mutated molecules is subjected to selection or screening procedures. In the latter, discrete regions of a polypeptide, corresponding either to defined structural or functional determinants (e.g., the extracellular, transmembrane, or cytoplasmic domain) are subjected to saturating or semi-random mutagenesis and these mutagenized cassettes are re-introduced into the context of the otherwise wild type allele. In one embodiment, PCR mutagenesis can be used. For example, Megaprimer PCR can be used (O. H. Landt, 1990. Gene 96:125-128).

In addition to full length NIMR polypeptides, fragments of NIMR polypeptides and their use are also within the scope of the invention. As used herein, a fragment of an NIMR polypeptide refers to a portion of a full-length NIMR polypeptide which is useful in a screening assay to identify compounds which modulate a biological activity of an NIMR polypeptide (e.g., alter the ability of an NIMR polypeptide to influence drug resistance in a microbe). Accordingly, isolated NIMR polypeptides for use in the instant screening assays can be full length NIMR polypeptides or fragments thereof. Thus, an isolated NIMR polypeptide can comprise, consist essentially of, or consist of an amino acid sequence derived from the full length amino acid sequence of an NIMR polypeptide, provided that it retains an NIMR polypeptide activity.

Portions of the above described polypeptide suitable for use in the claimed assays, such as those which retain their function (e.g., the ability to modulate drug resistance, the ability to modulate drug efflux from a cell, or those which are critical for binding to other molecules (such as DNA, proteins, or compounds) can be easily determined by one of ordinary skill in the art, e.g, using standard truncation or mutagenesis techniques and used in the instant assays. Exemplary techniques are described by Gallegos et al. (1996. J. Bacteriol. 178:6427). In addition, biologically active portions of an NIMR polypeptide include peptides comprising amino acid sequences sufficiently homologous to or derived from the amino acid sequence of the NIMR polypeptide, which include fewer amino acids than the full length NIMR polypeptides, and exhibit at least one activity of an NIMR polypeptide are also the subject of the invention.

Other fragments include, for example, truncation polypeptides having a portion of an amino acid sequence of an NIMR molecule shown in Table 1, or of variants thereof, such as a continuous series of residues that includes the amino terminus, or a continuous series of residues that includes the carboxyl terminus. Degradation forms of the polypeptides of the invention in a host cell are also preferred. Further preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which are introduced for optimal alignment of the two sequences. As used herein amino acid or nucleic acid “identity” is equivalent to amino acid or nucleic acid “homology”.

The comparison of sequences and determination of percent identity between two sequences can be accomplished using a mathematical algorithm. A non-limiting example of a mathematical algorithm utilized for comparison of sequences is the algorithm of Karlin and Altschul (1990) Proc. Natl. Acad. Sci. USA 87:2264, modified as in Karlin and Altschul (1993) Proc. Natl. Acad. Sci. USA 90:5873. Such an algorithm is incorporated into the NBLAST and XBLAST programs (version 2.0) of Altschul, et al. (1990) J. Mol. Biol. 215:403. BLAST nucleotide searches can be performed with the NBLAST program score=100, wordlength=12 to obtain nucleotide sequences homologous to the nucleic acid molecules of the invention. BLAST polypeptide searches can be performed with the XBLAST program, score=50, wordlength=3 to obtain amino acid sequences homologous to the polypeptide molecules of the invention. To obtain gapped alignments for comparison purposes, Gapped BLAST can be utilized as described in Altschul et al., (1997) Nucleic Acids Research 25(Hamilton, C. M., Aldea, M., Washburn, B. K., Babitzke, P. & Kushner, S. R. (1989) J. Bacteriol. 171, 4617-4622):3389. When utilizing BLAST and Gapped BLAST programs, the default parameters of the respective programs (e.g., XBLAST and NBLAST) can be used. See http://www.ncbi.nlm.nih.gov. Another preferred, non-limiting algorithm utilized for the comparison of sequences is the algorithm of Myers and Miller, CABIOS (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Another non-limiting example of a mathematical algorithm utilized for the alignment of polypeptide sequences is the Lipman-Pearson algorithm (Lipman and Pearson (1985) Science 227:1435). When using the Lipman-Pearson algorithm, a PAM250 weight residue table, a gap length penalty of 12, a gap penalty of 4, and a Kutple of 2 can be used. A preferred, non-limiting example of a mathematical algorithm utilized for the alignment of nucleic acid sequences is the Wilbur-Lipman algorithm (Wilbur and Lipman (1983) Proc. Natl. Acad. Sci. USA 80:726). When using the Wilbur-Lipman algorithm, a window of 20, gap penalty of 3, Ktuple of 3 can be used. Both the Lipman-Pearson algorithm and the Wilbur-Lipman algorithm are incorporated, for example, into the MEGALIGN program (e.g., version 3.1.7) which is part of the DNASTAR sequence analysis software package.

Additional algorithms for sequence analysis are known in the art, and include ADVANCE and ADAM., described in Torelli and Robotti (1994) Comput. Appl. Biosci. 10:3; and FASTA, described in Pearson and Lipman (1988) PNAS 85:2444.

In a preferred embodiment, the percent identity between two amino acid sequences is determined using the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6.

Protein alignments can also be made using the Geneworks global polypeptide alignment program (e.g., version 2.5.1) with the cost to open gap set at 5, the cost to lengthen gap set at 5, the minimum diagonal length set at 4, the maximum diagonal offset set at 130, the consensus cutoff set at 50% and utilizing the Pam 250 matrix.

The nucleic acid and polypeptide sequences of the present invention can further be used as a “query sequence” to perform a search against public databases to, for example, identify other members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to NIMR nucleic acid molecules of the invention. BLAST polypeptide searches can be performed with the XBLAST program, score=50, wordlength=3 to obtain amino acid sequences homologous to NIMR polypeptide molecules of the invention. To obtain gapped alignments for comparison purposes, Gapped BLAST can be utilized as described in Altschul et al., (1997) Nucleic Acids Res. 25(Hamilton, C. M., Aldea, M., Washburn, B. K., Babitzke, P. & Kushner, S. R. (1989) J. Bacteriol. 171, 4617-4622):3389-3402. When utilizing BLAST and Gapped BLAST programs, the default parameters of the respective programs (e.g., XBLAST and NBLAST) can be used. For example, the nucleotide sequences of the invention can be analyzed using the default Blastn matrix 1-3 with gap penalties set at: existence 11 and extension 1. The amino acid sequences of the invention can be analyzed using the default settings: the Blosum62 matrix with gap penalties set at existence 11 and extension 1. See http://www.ncbi.nlm.nih.gov.

The invention also provides NIMR chimeric or fusion polypeptides. As used herein, an NIMR “chimeric polypeptide” or “fusion polypeptide” comprises an NIMR polypeptide operatively linked to a non-NIMR polypeptide. An “NIMR polypeptide” refers to a polypeptide having an amino acid sequence corresponding to NIMR polypeptide, whereas a “non-NIMR polypeptide” refers to a polypeptide having an amino acid sequence corresponding to a polypeptide which is not substantially homologous to the NIMR polypeptide, e.g., a polypeptide which is different from the NIMR polypeptide and which is derived from the same or a different organism. Within an NIMR fusion polypeptide the NIMR polypeptide can correspond to all or a portion of an NIMR polypeptide. In a preferred embodiment, an NIMR fusion polypeptide comprises at least one biologically active portion of an NIMR polypeptide. Within the fusion polypeptide, the term “operatively linked” is intended to indicate that the NIMR polypeptide and the non-NIMR polypeptide are fused in-frame to each other. The non-NIMR polypeptide can be fused to the N-terminus or C-terminus of the NIMR polypeptide.

For example, in one embodiment, the fusion polypeptide is a GST-NIMR member fusion polypeptide in which the NIMR member sequences are fused to the C-terminus of the GST sequences. In another embodiment, the fusion polypeptide is an NIMR-HA fusion polypeptide in which the NIMR member nucleotide sequence is inserted in a vector such as pCEP4-HA vector (Herrscher, R. F. et al. (1995) Genes Dev. 9:3067-3082) such that the NIMR member sequences are fused in frame to an influenza hemagglutinin epitope tag. Such fusion polypeptides can facilitate the purification of a recombinant NIMR polypeptide.

Fusion polypeptides and peptides produced by recombinant techniques may be secreted and isolated from a mixture of cells and medium containing the polypeptide or peptide. Alternatively, the polypeptide or peptide may be retained cytoplasmically and the cells harvested, lysed and the polypeptide isolated. A cell culture typically includes host cells, media and other byproducts. Suitable media for cell culture are well known in the art. Polypeptides can be isolated from cell culture media, host cells, or both using techniques known in the art for purifying polypeptides and peptides. Techniques for transfecting host cells and purifying polypeptides and peptides are known in the art.

Preferably, an NIMR fusion polypeptide of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A NIMR encoding nucleic acid molecule can be cloned into such an expression vector such that the fusion moiety is linked in-frame to the NIMR polypeptide.

In another embodiment, the fusion polypeptide is an NIMR polypeptide containing a heterologous signal sequence at its N-terminus. In certain host cells (e.g., mammalian host cells), expression and/or secretion of NIMR can be increased through use of a heterologous signal sequence. The NIMR fusion polypeptides of the invention can be incorporated into pharmaceutical compositions and administered to a subject in vivo. Use of NIMR fusion polypeptides may be useful therapeutically for the treatment of infection. Moreover, the NIMR-fusion polypeptides of the invention can be used as immunogens to produce anti-NIMR antibodies in a subject.

Preferably, an NIMR chimeric or fusion polypeptide of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A NIMR-encoding nucleic acid can be cloned into such an expression vector such that the fusion moiety is linked in-frame to the NIMR polypeptide.

The present invention also pertains to variants of the NIMR polypeptides which function as either NIMR agonists (mimetics) or as NIMR antagonists. Variants of the NIMR polypeptides can be generated by mutagenesis, e.g., discrete point mutation or truncation of an NIMR polypeptide. An agonist of the NIMR polypeptides can retain substantially the same, or a subset, of the biological activities of the naturally occurring form of an NIMR polypeptide. An antagonist of an NIMR polypeptide can inhibit one or more of the activities of the naturally occurring form of the NIMR polypeptide by, for example, competitively modulating a cellular activity of an NIMR polypeptide. Thus, specific biological effects can be elicited by treatment with a variant of limited function. In one embodiment, treatment of a subject with a variant having a subset of the biological activities of the naturally occurring form of the polypeptide has fewer side effects in a subject relative to treatment with the naturally occurring form of the NIMR polypeptide.

In one embodiment, the invention pertains to derivatives of NIMR which may be formed by modifying at least one amino acid residue of NIMR by oxidation, reduction, or other derivatization processes known in the art.

In one embodiment, variants of an NIMR polypeptide which function as either NIMR agonists (mimetics) or as NIMR antagonists can be identified by screening combinatorial libraries of mutants, e.g., truncation mutants, of an NIMR polypeptide for NIMR polypeptide agonist or antagonist activity. In one embodiment, a variegated library of NIMR variants is generated by combinatorial mutagenesis at the nucleic acid level and is encoded by a variegated gene library. A variegated library of NIMR variants can be produced by, for example, enzymatically ligating a mixture of synthetic oligonucleotides into gene sequences such that a degenerate set of potential NIMR sequences is expressible as individual polypeptides, or alternatively, as a set of larger fusion polypeptides (e.g., for phage display) containing the set of NIMR sequences therein. There are a variety of methods which can be used to produce libraries of potential NIMR variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NIMR sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In one embodiment, a library of coding sequence fragments can be generated by treating a double stranded PCR fragment of an NIMR coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the NIMR polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NIMR polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify NIMR variants (Arkin and Yourvan (1992) Proc. Natl. Acad. Sci. USA 89:7811-7815; Delgrave et al. (1993) Protein Engineering 6(Cohen, S. P., Hachler, H. & Levy, S. B. (1993) J. Bacteriol. 175, 1484-1492):327-331).

In one embodiment, cell based assays can be exploited to analyze a variegated NIMR library. For example, a library of expression vectors can be transfected into a cell line which ordinarily synthesizes and secretes NIMR. The transfected cells are then cultured such that NIMR and a particular mutant NIMR are secreted and the effect of expression of the mutant on NIMR activity in cell supernatants can be detected, e.g., by any of a number of enzymatic assays. Plasmid DNA can then be recovered from the cells which score for inhibition, or alternatively, potentiation of NIMR activity, and the individual clones further characterized.

In addition to NIMR polypeptides comprising only naturally-occurring amino acids, NIMR peptidomimetics are also provided. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15: 29; Veber and Freidinger (1985) TINS p.392; and Evans et al. (1987) J. Med. Chem 30: 1229, which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), such as NIMR, but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., Int J Pept Prot Res (1979) 14:177-185 (—CH2NH—, CH2CH2-); Spatola, A. F. et al., Life Sci (1986) 38:1243-1249 (—CH2-S); Hann, M. M., J Chem Soc Perkin Trans 1 (1982) 307-314 (—CH—CH—, cis and trans); Almquist, R. G. et al., J Med Chem (1980) 23:1392-1398 (—COCH2-); Jennings-White, C. et al., Tetrahedron Lett (1982) 23:2533 (—COCH2-); Szelke, M. et al., European Appln. EP 45665 (1982) CA: 97:39405 (1982)(—CH(OH)CH2-); Holladay, M. W. et al., Tetrahedron Lett (1983) 24:4401-4404 (—C(OH)CH2-); and Hruby, V. J., Life Sci (1982) 31:189-199 (—CH2-S—); each of which is incorporated herein by reference. A particularly preferred non-peptide linkage is —CH2NH—.

Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally, are positions that do not form direct contacts with the macromolecules(s) to which the peptidomimetic binds to produce the therapeutic effect. Derivitization (e.g., labelling) of peptidomimetics should not substantially interfere with the desired biological or pharmacological activity of the peptidomimetic.

Systematic substitution of one or more amino acids of an NIMR amino acid sequence with a D-amino acid of the same type (e.g., D-lysine in place of L-lysine) may be used to generate more stable peptides. In addition, constrained peptides comprising an NIMR amino acid sequence or a substantially identical sequence variation may be generated by methods known in the art (Rizo and Gierasch (1992) Ann. Rev. Biochem. 61: 387, incorporated herein by reference); for example, by adding internal cysteine residues capable of forming intramolecular disulfide bridges which cyclize the peptide.

The amino acid sequences of NIMR polypeptides identified herein will enable those of skill in the art to produce polypeptides corresponding to NIMR peptide sequences and sequence variants thereof. Such polypeptides may be produced in prokaryotic or eukaryotic host cells by expression of polynucleotides encoding an NIMR peptide sequence, frequently as part of a larger polypeptide. Alternatively, such peptides may be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91: 501; Chaiken I. M. (1981) CRC Crit. Rev. Biochem. 11: 255; Kaiser et al. (1989) Science 243: 187; Merrifield, B. (1986) Science 232: 342; Kent, S. B. H. (1988) Ann. Rev. Biochem. 57: 957; and Offord, R. E. (1980) Semisynthetic Proteins, Wiley Publishing, which are incorporated herein by reference).

Peptides can be produced, typically by direct chemical synthesis, and used e.g., as agonists or antagonists of an NIMR molecule, e.g., to modulate binding of an NIMR polypeptide and a molecule with which it normally interacts. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides may be used therapeutically, e.g, to treat infection.

An isolated NIMR polypeptide, or a portion or fragment thereof, can also be used as an immunogen to generate antibodies that bind NIMR using standard techniques for polyclonal and monoclonal antibody preparation. A full-length NIMR polypeptide can be used or, alternatively, the invention provides antigenic peptide fragments of NIMR for use as immunogens. The antigenic peptide of NIMR preferably comprises at least 8 amino acid residues and encompasses an epitope of NIMR such that an antibody raised against the peptide forms a specific immune complex with NIMR. More preferably, the antigenic peptide comprises at least 10 amino acid residues, even more preferably at least 15 amino acid residues, even more preferably at least 20 amino acid residues, and most preferably at least 30 amino acid residues.

Alternatively, an antigenic peptide fragment of an NIMR polypeptide can be used as the immunogen. An antigenic peptide fragment of an NIMR polypeptide typically comprises at least 8 amino acid residues of an amino acid sequence of an NIMR molecule of Table 1 and encompasses an epitope of an NIMR polypeptide such that an antibody raised against the peptide forms an immune complex with an NIMR molecule. Preferred epitopes encompassed by the antigenic peptide are regions of NIMR that are located on the surface of the polypeptide, e.g., hydrophilic regions. In one embodiment, an antibody binds substantially specifically to an NIMR polypeptide. In another embodiment, an antibody binds specifically to an NIMR polypeptide.

In one embodiment such epitopes can be specific for an NIMR polypeptide from one species (i.e., an antigenic peptide that spans a region of an NIMR polypeptide that is not conserved across species is used as immunogen; such non conserved residues can be determined using an alignment such as that provided herein). A standard hydrophobicity analysis of the polypeptide can be performed to identify hydrophilic regions.

Accordingly, another aspect of the invention pertains to the use of anti-NIMR antibodies. Polyclonal anti-NIMR antibodies can be prepared as described above by immunizing a suitable subject with an NIMR immunogen. The anti-NIMR antibody titer in the immunized subject can be monitored over time by standard techniques, such as with an enzyme linked immunosorbent assay (ELISA) using immobilized an NIMR polypeptide. If desired, the antibody molecules directed against an NIMR polypeptide can be isolated from the mammal (e.g., from the blood) and further purified by well known techniques, such as polypeptide A chromatography to obtain the IgG fraction. At an appropriate time after immunization, e.g., when the anti-NIMR antibody titers are highest, antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J Immunol 127:539-46; Brown et al. (1980) J Biol Chem 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma. technique (Cole et al. (1985), Monoclonal Antibodies and Cancer Therapy, Alan R. Liss, Inc., pp. 77-96) or trioma techniques.

As an alternative to preparing monoclonal antibody-secreting hybridomas, a monoclonal anti-NIMR antibody can be identified and isolated by screening a recombinant combinatorial immunoglobulin library (e.g., an antibody phage display library) with an NIMR to thereby isolate immunoglobulin library members that bind an NIMR polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Additionally, recombinant anti-NIMR antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira, et al European Patent Application 184, 187; Taniguchi, M., European Patent Application 171, 496; Morrison et al. European Patent Application 173, 494; Neuberger et al. PCT Application WO 86/01533; Cabilly et al. U.S. Pat. No. 4,816,567; Cabilly et al. European Patent Application 125, 023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-NIMR antibody (e.g., monoclonal antibody) can be used to isolate an NIMR polypeptide by standard techniques, such as affinity chromatography or immunoprecipitation. Anti-NIMR antibodies can facilitate the purification of natural NIMR polypeptides from cells and of recombinantly produced NIMR polypeptides expressed in host cells. Moreover, an anti-NIMR antibody can be used to detect an NIMR polypeptide (e.g., in a cellular lysate or cell supernatant). Detection may be facilitated by coupling (i.e., physically linking) the antibody to a detectable substance. Accordingly, in one embodiment, an anti-NIMR antibody of the invention is labeled with a detectable substance. Examples of detectable substances include various enzymes, prosthetic groups, fluorescent materials, luminescent materials and radioactive materials.

III. Microbes

Numerous different microbes are suitable for use as sources of NIMR nucleic acid molecules or polypeptides, as host cells, and in testing for compounds in the screening assays described herein, e.g., for testing for compounds that modulate the activity and/or expression of an NIMR polypeptides. The term “microbe” includes microorganisms having an NIMR polypeptide or those that can be engineered to express such a molecule for the purposes of developing a screening assay. Preferably “microbe” refers to unicellular prokaryotic or eukaryotic microbes including bacteria, fungi, or protozoa. In another embodiment, microbes suitable for use in the invention are multicellular, e.g., parasites or fungi. In preferred embodiments, microbes are pathogenic for humans, animals, or plants. In other embodiments, microbes causing environmental problems, e.g., fouling or spoilage or that perform useful functions such as breakdown of plant matter are also preferred. As such, any of these disclosed microbes may be used as intact cells or as sources of materials for cell-free assays as described herein.

In preferred embodiments, microbes for use in the claimed methods are bacteria, either Gram-negative or Gram-positive bacteria. In a preferred embodiment, any bacteria that are shown to become resistant to drugs, preferably antibiotics, are appropriate for use in the claimed methods.

In preferred embodiments, microbes are bacteria from the family Enterobacteriaceae. In more preferred embodiments bacteria of a genus selected from the group consisting of: Escherichia, Proteus, Salmonella, Klebsiella, Shigella, Providencia, Enterobacter, Burkholderia, Pseudomonas, Acinetobacter, Aeromonas, Haemophilus, Yersinia, Neisseria, and Erwinia, Rhodopseudomonas, or Burkholderia.

In yet other embodiments, the microbes are Gram-positive bacteria and are from a genus selected from the group consisting of: Lactobacillus, Azorhizobium, Streptomyces, Pediococcus, Photobacterium, Bacillus, Enterococcus, Staphylococcus, Clostridium, Streptococcus, Butyrivibrio, Sphingomonas, Rhodococcus, or Streptomyces

In yet other embodiments, the microbes are acid fast bacilli, e.g., from the genus Mycobacterium.

In still other embodiments, the microbes are, e.g., selected from a genus selected from the group consisting of: Methanobacterium, Sulfolobus, Archaeoglobu, Rhodobacter, or Sinorhizobium.

In other embodiments, the microbes are fungi. In a preferred embodiment the fungus is from the genus Mucor or Candida, e.g., Mucor racemosus or Candida albicans.

In yet other embodiments, the microbes are protozoa. In a preferred embodiment the microbe is a malaria or cryptosporidium parasite.

IV. Vectors and Host Cells

Preferred NIMR polypeptides for use in screening assays are “isolated” or recombinant polypeptides. In one embodiment, NIMR polypeptides can be made from isolated nucleic acid molecules. Nucleic acid molecules encoding NIMR polypeptides can be used for screening or can be used to produce NIMR polypeptides for use in the instant assays. For example, nucleic acid molecules encoding an NIMR polypeptide can be isolated (e.g., isolated from the sequences which naturally flank it in the chromosome and from cellular components) and can be used to produce an NIMR polypeptide. In one embodiment; a nucleic acid molecule which has been (George, A. M. & Levy, S. B. (1983) J. Bacteriol. 155, 541-548) amplified in vitro by, for example, polymerase chain reaction (PCR); (Cohen, S. P., Yan, W. & Levy, S. B. (1993) J. Infect. Dis. 168, 484-488) recombinantly produced by cloning, or (Cohen, S. P., Hachler, H. & Levy, S. B. (1993) J. Bacteriol. 175, 1484-1492) purified, as by cleavage and gel separation; or (Sulavick, M. C., Dazer, M. & Miller, P. F. (1997) J. Bacteriol. 179, 1857-1866) synthesized by, for example, chemical synthesis can be used to produce NIMR polypeptides.

NIMR polypeptides can be expressed in a modified form. For example, for secretion of the translated polypeptide into the lumen of the endoplasmic reticulum, into the periplasmic space or into the extracellular environment, appropriate secretion signals may be incorporated into the expressed polypeptide. These signals may be endogenous to the polypeptide or they may be heterologous signals. Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding proteins may be employed to regenerate active conformation when the polypeptide is denatured during isolation and or purification.

For recombinant production, host cells can be genetically engineered to incorporate nucleic acid molecules of the invention. In one embodiment nucleic acid molecules specifying NIMR polypeptides can be placed in a vector. The term “vector” refers to a nucleic acid molecule capable of transporting another nucleic acid molecule to which it has been linked. The term “expression vector” or “expression system” includes any vector, (e.g., a plasmid, cosmid or phage chromosome) containing a gene construct in a form suitable for expression by a cell (e.g., linked to a promoter). In the present specification, “plasmid” and “vector” are used interchangeably, as a plasmid is a commonly used form of vector. Moreover, the invention is intended to include other vectors which serve equivalent functions. A great variety of expression systems can be used to produce the polypeptides of the invention. Such vectors, include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Appropriate vectors are widely available commercially and it is within the knowledge and discretion of one of ordinary skill in the art to choose a vector which is appropriate for use with a given host cell. The sequences encoding NIMR polypeptides can be introduced into a cell on a self-replicating vector or may be introduced into the chromosome of a microbe using homologous recombination or by an insertion element such as a transposon.

The expression system constructs may contain control regions that regulate expression. “Transcriptional regulatory sequence” is a generic term to refer to DNA sequences, such as initiation signals, enhancers, operators, and promoters, which induce or control transcription of polypeptide coding sequences with which they are operably linked. It will also be understood that a recombinant gene encoding an NIMR polypeptide can be under the control of transcriptional regulatory sequences which are the same or which are different from those sequences which control transcription of the naturally-occurring NIMR gene. Exemplary regulatory sequences are described in Goeddel; Gene Expression Technology: Methods in Enzymology 185, Academic Press, San Diego, Calif. (1990). For instance, any of a wide variety of expression control sequences, that control the expression of a DNA sequence when operatively linked to it, may be used in these vectors to express DNA sequences encoding the NIMR polypeptides of this invention.

Generally, any system or vector suitable to maintain, propagate or express nucleic acid molecules and/or to express a polypeptide in a host may be used for expression in this regard. The appropriate DNA sequence may be inserted into the expression system by any of a variety of well-known and routine techniques, such as, for example, those set forth in Sambrook et al., Molecular Cloning, A Laboratory Manual, (supra).

Exemplary expression vectors for expression of a gene encoding an NIMR polypeptide and capable of replication in a bacterium, e.g., a gram positive, gram negative, or in a cell of a simple eukaryotic fungus such as a Saccharomyces or, Pichia, or in a cell of a eukaryotic organism such as an insect, a bird, a mammal, or a plant, are known in the art. Such vectors may carry functional replication-specifying sequences (replicons) both for a host for expression, for example a Streptomyces, and for a host, for example, E. coli, for genetic manipulations and vector construction. See e.g. U.S. Pat. No. 4,745,056. Suitable vectors for a variety of organisms are described in Ausubel, F. et al., Short Protocols in Molecular Biology, Wiley, New York (1995), and for example, for Pichia, can be obtained from Invitrogen (Carlsbad, Calif.).

Useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat polypeptide, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. A useful translational enhancer sequence is described in U.S. Pat. No. 4,820,639.

It should be understood that the design of the expression vector may depend on such factors as the choice of the host cell to be transformed and/or the type of polypeptide desired to be expressed. Representative examples of appropriate hosts include bacterial cells, such as gram positive, gram negative cells; fungal cells, such as yeast cells and Aspergillus cells; insect cells such as Drosophila S2 and Spodoplera Sf9 cells; animal cells such as CHO, COS, HeLa, C127, 3T3, BHK, 293 and Bowes melanoma cells; and plant cells.

In preferred embodiments, cells used to express NIMR polypeptides for purification or for use in screening assays, e.g., host cells, comprise a mutation which renders any endogenous NIMR polypeptide nonfunctional or causes the endogenous polypeptide to not be expressed. In other embodiments, mutations may also be made in other related genes of the host cell, such that there will be no interference from the endogenous host loci.

Introduction of a nucleic acid molecule into the host cell (“transformation”) can be effected by methods described in many standard laboratory manuals, such as Davis et al., Basic Methods In Molecular Biology, (1986) and Sambrook et al., Molecular Cloning: A Laboratory Manual, 2nd Ed., Cold Spring Harbor Laboratory Press, Cold Spring Harbor, N.Y. (1989). Examples include electroporation, phosphate transfection, DEAE-dextran mediated transfection, transvection, microinjection, cationic lipid-mediated transfection, electroporation, transduction, scrape loading, ballistic introduction and infection.

Purification of an NIMR polypeptides, e.g., recombinantly expressed polypeptides, can be accomplished using techniques known in the art. For example, if the NIMR polypeptide is expressed in a form that is secreted from cells, the medium can be collected. Alternatively, if the NIMR polypeptide is expressed in a form that is retained by cells, the host cells can be lysed to release the NIMR polypeptide. Such spent medium or cell lysate can be used to concentrate and purify the NIMR polypeptide. For example, the medium or lysate can be passed over a column, e.g., a column to which antibodies specific for the NIMR polypeptide have been bound. Alternatively, such antibodies can be specific for a non-NIMR polypeptide which has been fused to the NIMR polypeptide (e.g., as a tag) to facilitate purification of the NIMR polypeptide. Other means of purifying NIMR polypeptides are known in the art.

V. Uses of NIMR Compositions

The NIMR modulating agents (e.g., nucleic acid molecules, polypeptides, variants, polypeptide homologues, NIMR agonists or antagonists, and antibodies described herein) can be used in one or more of the following methods: a) methods of treatment, e.g., a) treatment of infection and disinfection of surfaces; b) screening assays; c) use in vaccines, d) diagnostic assays, and the like. The isolated nucleic acid molecules of the invention can be used, for example, to express NIMR polypeptide (e.g., in a host cell in gene therapy applications), to detect NIMR mRNA (e.g., in a biological sample) or a genetic alteration in an NIMR gene, and to modulate NIMR activity, as described further below. In addition, the NIMR polypeptides can be used, e.g., to screen for naturally occurring NIMR binding polypeptides, to screen for drugs or compounds which modulate NIMR activity (e.g., are agonists or antagonists of NIMR activity), as well as to treat disorders that would benefit from modulation of NIMR, e.g., infection with a microbe. The NIMR modulating agents can be used to treat infection (e.g., alone or in combination with a second drug, e.g., an antibiotic) or to reduce contamination (e.g., alone or in combination with a non-antibiotic agent). NIMR modulating agents can also be used to alter MarA regulation of NIMR genes. For example, such agents can be used to downregulate genes that are normally upregulated by MarA or to upregulate genes that are normally downregulated by MarA. Moreover, the anti-NIMR antibodies of the invention can be used to modulate NIMR activity and to detect and isolate NIMR polypeptides, regulate the bioavailability of NIMR polypeptides, and modulate NIMR activity.

A. Methods of Treatment

The subject compositions can be used in treating disorders that would benefit from modulation of an NIMR polypeptide activity, e.g., in treating a subject having an infection with a microbe.

As used herein the term “infection” includes the presence of a microbe in or on a subject which, if its growth were inhibited, would result in a benefit to the subject. As such, the term “infection” in addition to referring to the presence of pathogens also includes normal flora which is not desirable, e.g., on the skin of a burn patient or in the gastrointestinal tract of an immunocompromised patient. As used herein, the term “treating” refers to the administration of a compound to a subject, for prophylactic and/or therapeutic purposes. The term “administration” includes delivery to a subject, e.g., by any appropriate method which serves to deliver the drug to the site of the infection. Administration of the drug can be, e.g., oral, intravenous, or topical (as described in further detail below). Drugs can also be contacted with microbes that are not present in the body, but are present in the environment, e.g., on surfaces.

Methods of modulating expression and/or activity of an NIMR polypeptide in a microbial cell are useful in modulation, e.g., of microbial adapatation to environmental stress and/or moduation of microbial virulence. Generally, it is desirable to increase expression and/or activity of those genes that are downmodulated by overexpression of MarA and to decrease the expression and/or activity of those genes that are upmodulated by overexpression of MarA.

Exemplary NIMR downmodulatory agents include: antisense NIMR nucleic acid molecules, anti-NIMR antibodies, dominant negative NIMR mutants, NIMR antagonists, or compounds which downmodulate NIMR activity identified using the subject screening assays. Additionally or alternatively, compounds which downmodulate NIMR activity can be designed using approaches known in the art.

Exemplary NIMR stimulatory agents include active NIMR polypeptide molecules and nucleic acid molecules encoding NIMR that are introduced into a cell to increase NIMR activity in the cell.

The modulatory methods of the invention can be performed in vitro or in vivo.

NIMR modulating agents can be used alone, in combination with other NIMR modulating agents (e.g., that modulate the same or a different NIMR molecule), or with other drugs (e.g., antibiotic or non-antibiotic drugs).

In one embodiment, an NIMR modulating agent can be administered to a subject alone, e.g., prior to administration of an antibiotic agent in order to increase the efficacy of the antibiotic. In one embodiment, an NIMR modulating agent can be administered to a subject in combination with an antibiotic agent in order to increase the efficacy of the antibiotic.

In another embodiment, an NIMR modulating agent or agents can be used to disinfect surfaces, e.g., in combination with a non-antibiotic agent such as a biocide, in order to increase the effectiveness of the non-antibiotic agent.

In one embodiment, a “combination product” can be formulated comprising an NIMR modulating agent and a non-antibiotic agent, e.g., a disinfectant for decontamination of surfaces or a consumer product (e.g., a detergent, soap, deodorant, mouthwash, toothpaste, or lotion).

B. Uses in Identifying NIMR Agonists and Antagonists

The invention provides a method (also referred to herein as a “screening assay”) to identify those which modulate (enhance (agonists) or block (antagonists)) the action of NIMR polypeptides or nucleic acid molecules, particularly those compounds that are bacteriostatic and/or bactericidal or prevent the infectious process. The subject screening assays can be used to identify modulators, i.e., candidate or test compounds or agents (e.g., peptides, peptidomimetics, small molecules or other drugs) which modulate NIMR polypeptides, i.e., have a stimulatory or inhibitory effect on, for example, NIMR polypeptide expression or NIMR polypeptide activity. Test compounds may be natural substrates and ligands or may be structural or functional mimetics. See, e.g., Coligan et al., Current Protocols in Immunology 1(2): Chapter 5 (1991).

NIMR polypeptide agonists and antagonists can be assayed in a variety of ways. For example, in one embodiment, the invention provides for methods for identifying a compound which modulates an NIMR molecule, e.g., by measuring the ability of the compound to interact with an NIMR nucleic acid molecule or an NIMR polypeptide or the ability of a compound to modulate the activity or expression of an NIMR polypeptide. Furthermore, the ability of a compound to modulate the binding of an NIMR polypeptide or NIMR nucleic acid molecule to a molecule to which they normally bind, e.g., an NIMR binding polypeptide can be tested.

Compounds for testing in the instant methods can be derived from a variety of different sources and can be known or can be novel. Preferably, a screening assay is performed to test the activity of a compound not previously known to have the activity tested for. Each of the NIMR sequences provided herein may be used in the discovery and development of antibacterial compounds. The NIMR polypeptide or portions thereof, upon expression, can be used as a target for the screening of antibacterial drugs. In another embodiment, antisense nucleic acid molecules or nucleic acid molecules that encode for dominant negative NIMR mutants can also be tested in the subject assays.

In one embodiment, libraries of compounds are tested in the instant methods. In another embodiment, known compounds are tested in the instant methods. In another embodiment, compounds among the list of compounds generally regarded as safe (GRAS) by the Environmental Protection Agency are tested in the instant methods.

In one embodiment, a library of compounds can be screened in the subject assays. A recent trend in medicinal chemistry includes the production of mixtures of compounds, referred to as libraries. While the use of libraries of peptides is well established in the art, new techniques have been developed which have allowed the production of mixtures of other compounds, such as benzodiazepines (Bunin et al. 1992. J. Am. Chem. Soc. 114:10987; DeWitt et al. 1993. Proc. Natl. Acad. Sci. USA 90:6909) peptoids (Zuckermann. 1994. J. Med Chem. 37:2678) oligocarbamates (Cho et al. 1993. Science. 261:1303), and hydantoins (DeWitt et al. supra). Rebek et al. have described an approach for the synthesis of molecular libraries of small organic molecules with a diversity of 10⁴-10⁵ (Carell et al. 1994. Angew. Chem. Int. Ed. Engl. 33:2059; Carell et al. Angew. Chem. Int. Ed. Engl. 1994. 33:2061).

The compounds for screening in the assays of the present invention can be obtained using any of the numerous approaches in combinatorial library methods known in the art, including: biological libraries; spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, non-peptide oligomer or small molecule libraries of compounds (Lam, K. S. Anticancer Drug Des. 1997. 12:145).

Exemplary compounds which can be screened for activity include, but are not limited to, peptides, nucleic acids, carbohydrates, small organic molecules (e.g., polyketides) (Cane et al. 1998. Science 282:63), and natural product extract libraries. In one embodiment, the test compound is a peptide or peptidomimetic. In another, preferred embodiment, the compounds are small, organic non-peptidic compounds.

Other exemplary methods for the synthesis of molecular libraries can be found in the art, for example in: Erb et al. 1994. Proc. Natl. Acad. Sci. USA 91:11422; Horwell et al. 1996 Immunopharmacology 33:68; and in Gallop et al. 1994. J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra). Other types of peptide libraries may also be expressed, see, for example, U.S. Pat. Nos. 5,270,181 and 5,292,646). In still another embodiment, combinatorial polypeptides can be produced from a cDNA library.

The efficacy of the agonist or antagonist can be assessed by generating dose response curves from data obtained using various concentrations of the test modulating agent. Moreover, a control assay can also be performed to provide a baseline for comparison. As described in more detail below, either whole cell or cell free assay systems can be employed.

1. Whole Cell Assays

In one embodiment of the invention, the subject screening assays can be performed using whole cells. In one embodiment of the invention, the step of determining whether a compound reduces the activity or expression of an NIMR polypeptide comprises contacting a cell expressing an NIMR polypeptide with a compound and measuring the ability of the compound to modulate the activity or expression of an NIMR polypeptide.

In another embodiment, modulators of NIMR polypeptide expression are identified in a method wherein a cell is contacted with a candidate compound and the expression of NIMR polypeptide mRNA or protein in the cell is determined. The level of expression of NIMR polypeptide mRNA or protein in the presence of the candidate compound is compared to the level of expression of NIMR polypeptide mRNA or polypeptide in the absence of the candidate compound. The candidate compound can then be identified as a modulator of NIMR polypeptide expression based on this comparison. For example, when expression of NIMR polypeptide mRNA or protein is greater (e.g., statistically significantly greater) in the presence of the candidate compound than in its absence, the candidate compound is identified as a stimulator of NIMR polypeptide mRNA or protein expression. Alternatively, when expression of NIMR polypeptide mRNA or protein is less (e.g., statistically significantly less) in the presence of the candidate compound than in its absence, the candidate compound is identified as an inhibitor of NIMR mRNA or protein expression. The level of NIMR mRNA or protein expression in the cells can be determined by methods described herein for detecting NIMR mRNA or protein.

To measure expression of an NIMR polypeptide, transcription of an NIMR nucleic acid molecule gene can be measured in control cells which have not been treated with the compound and compared with that of test cells which have been treated with the compound. For example, cells which express endogenous NIMR polypeptides or which are engineered to express or overexpress recombinant NIMR polypeptides can be caused to express or overexpress a recombinant NIMR polypeptide in the presence and absence of a test modulating agent of interest, with the assay scoring for modulation in NIMR polypeptide responses by the target cell mediated by the test agent. For example, as with the cell-free assays, modulating agents which produce a change, e.g., a statistically significant change in NIMR polypeptide-dependent responses (either an increase or decrease) can be identified.

Recombinant expression vectors that can be used for expression of NIMR polypeptide are known in the art (see discussions above). In one embodiment, within the expression vector the NIMR polypeptide-coding sequences are operatively linked to regulatory sequences that allow for constitutive or inducible expression of NIMR polypeptide in the indicator cell(s). Use of a recombinant expression vector that allows for constitutive or inducible expression of NIMR polypeptide in a cell is preferred for identification of compounds that enhance or inhibit the activity of NIMR polypeptide. In an alternative embodiment, within the expression vector the NIMR polypeptide coding sequences are operatively linked to regulatory sequences of the endogenous NIMR polypeptide gene (i.e., the promoter regulatory region derived from the endogenous gene). Use of a recombinant expression vector in which NIMR polypeptide expression is controlled by the endogenous regulatory sequences is preferred for identification of compounds that enhance or inhibit the transcriptional expression of NIMR polypeptide.

In one embodiment, the level of transcription can be determined by measuring the amount of RNA produced by the cell. For example, the RNA can be isolated from cells which express an NIMR polypeptide and that have been incubated in the presence or absence of compound. Northern blots using probes specific for the sequences to be detected can then be performed using techniques known in the art. Numerous other, art-recognized techniques can be used. For example, western blot analysis can be used to test for NIMR. For example, in another embodiment, transcription of specific RNA molecules can be detected using the polymerase chain reaction, for example by making cDNA copies of the RNA transcript to be measured and amplifying and measuring them. In another embodiment, RNAse protection assays, such as S1 nuclease mapping or RNase mapping can be used to detect the level of transcription of a gene. In another embodiment, primer extension can be used.

In yet other embodiments, the ability of a compound to induce a change in transcription or translation of an NIMR polypeptide can be accomplished by measuring the amount of NIMR polypeptide produced by the cell. Polypeptides which can be detected include any polypeptides which are produced upon the activation of an NIMR responsive promoter, including, for example, both endogenous sequences and reporter gene sequences. In one embodiment, the amount of polypeptide made by a cell can be detected using an antibody against that polypeptide. In other embodiments, the activity of such a polypeptide can be measured.

In one embodiment, other sequences which are regulated by an NIMR promoter (e.g., a promoter having sequence identity with a promoter that regulates expression of an NIMR gene set forth in Table 1) can be detected. In one embodiment, sequences not normally regulated by an NIMR promoter can be assayed by linking them to a promoter that regulates transcription of an NIMR polypeptide.

In preferred embodiments, to provide a convenient readout of the transcription from an NIMR promoter, such a promoter is linked to a reporter gene, the transcription of which is readily detectable. For example, a bacterial cell, e.g., an E. coli cell, can be transformed as taught in Cohen et al. 1993. J. Bacteriol. 175:7856.

Examples of reporter genes include, but are not limited to, CAT (chloramphenicol acetyl transferase) (Alton and Vapnek (1979), Nature 282: 864-869) luciferase, and other enzyme detection systems, such as beta-galactosidase; firefly luciferase (de Wet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); PhoA, alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent polypeptide (U.S. Pat. No. 5,491,084; WO96/23898).

In yet another embodiment, the ability of a compound to modulate an NIMR polypeptide activity, (e,g., to modulate microbial responses to environmental stress and, thereby, modulate microbial adaptation to stress and/or microbial virulence) can be tested by measuring the ability of the compound to affect the ability of a microbe to adapt to a drug, e.g. by testing the ability of the microbe to grow in the presence of the drug. For example, the ability of a test compound to modulate the minimal inhibitory concentration (MIC) of the indicator compound can be tested. Such an assay can be performed using a standard methods, e.g., an antibiotic disc assay or an automated growth assay, e.g., using a system such as one commercially available from Viteck. In one embodiment, the method comprises detecting the ability of the compound to modulate growth of a microbe in the presence of one or more non-antibiotic agents. In another embodiment, the method comprises detecting the ability of the compound to modulate growth of a microbe in the presence of one or more antibiotics.

In another embodiment, the ability of a test compound to modulate the efflux of a drug from the cell can be tested. In this method, microbes are contacted with a test compound with or without an indicator compound (an indicator compound is one which is normally exported by the cell). The ability of a test compound to inhibit the activity of an efflux pump is demonstrated by determining whether the intracellular concentration of the test compound or the indicator compound (e.g., a drug or a dye) is elevated in the presence of the test compound. If the intracellular concentration of the indicator compound is increased in the presence of the test compound as compared to the intracellular concentration in the absence of the test compound, then the test compound can be identified as an inhibitor of an efflux pump. Thus, one can determine whether or not the test compound is an inhibitor of an efflux pump by showing that the test compound affects the ability of an efflux pump present in the microbe to export the indicator compound.

The “intracellular concentration” of an indicator compound includes the concentration of the indicator compound inside the outermost membrane of the microbe. The outermost membrane of the microbe can be, e.g., a cytoplasmic membrane. In the case of Gram-negative bacteria, the relevant “intracellular concentration” is the concentration in the cellular space in which the indicator compound localizes, e.g., the cellular space which contains a target of the indicator compound.

In one embodiment, the method comprises detecting the ability of the compound to reduce antibiotic resistance in a microbe. For example, in one embodiment, the indicator compound comprises an antibiotic and the effect of the test compound on the intracellular concentration of antibiotic in the microbe is measured. In one embodiment, an increase in the intracellular concentration of antibiotic can be measured directly, e.g., in an extract of microbial cells. For example, accumulation of a radiolabelled antibiotic can be determined using standard techniques. For instance, microbes can be contacted with a radiolabelled antibiotic as an indicator composition in the presence and absence of a test compound. The concentration of the antibiotic inside the cells can be measured at equilibrium by harvesting cells from the two groups (with and without test compound) and cell associated radioactivity measured with a liquid scintillation counter. In another embodiment, an increase in the intracellular concentration of antibiotic can be measured indirectly, e.g., by a showing that a given concentration of antibiotic when contacted with the microbe is sufficient to inhibit the growth of the microbe in the presence of the test compound, but not in the absence of the test compound.

In another embodiment, measurement of the intracellular concentration of an indicator compound can be facilitated by using an indicator compound which is readily detectable by spectroscopic means. Such a compound may be, for example, a dye, e.g., a basic dye, or a fluorophore. Exemplary indicator compounds include: acridine, ethidium bromode, gentian violet, malachite green, methylene blue, beenzyn viologen, bromothymol blue, toluidine blue, methylene blue rose bengal, alcyan blue, ruthenium red, fast green, aniline blue, xylene cyanol, bromophenol blue, coomassie blue, bormocresol purple, bromocresol green, trypan blue, and phenol red.

In such an assay, the effect of the test compound on the ability of the cell to export the indicator compound can be measured spectroscopically. For example, the intracellular concentration of the dye or fluorophore can be determined indirectly, by determining the concentration of the indicator compound in the suspension medium or by determining the concentration of the indicator compound in the cells. This can be done, e.g., by extracting the indicator compound from the cells or by visual inspection of the cells themselves.

In another embodiment, the presence of an indicator compound in a microbe can be detected using a reporter gene which is sensitive to the presence of the indicator compound. Exemplary reporter genes are known in the art. For example, a reporter gene can provide a colorometric read out or an enzymatic read out of the presence of an indicator compound. In yet another embodiment, a reporter gene whose expression is inducible by the presence of a drug in a microbe can be used. For example, a microbe can be grown in the presence of a drug with and without a putative test compound. In cells in which the efflux pump is inhibited, the concentration of the drug will be increased and the reporter gene construct will be expressed. By this method, efflux pump inhibitors are identified by their ability to inhibit the export rate of the drug and, thus, to induce reporter gene expression.

In another embodiment, a primary screening assay is used in which an indicator compound which does not comprise an antibiotic is employed. In one embodiment, upon the identification of a test compound that increases the intracellular concentration of the test compound, a secondary screening assay is performed in which the effect of the same test compound on susceptibility to the drug of interest, e.g., antibiotic resistance, is measured.

In yet another embodiment, the ability of a compound to modulate the binding of an NIMR polypeptide to an NIMR binding polypeptide can be determined. NIMR binding polypeptides can be identified using techniques which are known in the art. For example, in the case of binding polypeptides that interact with NIMR polypeptides, interaction trap assays or two hybrid screening assays can be used.

NIMR binding polypeptides can be identified e.g., e.g., by using an NIMR polypeptides or portions thereof of the invention as a “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with NIMR polypeptides (“NIMR-binding polypeptides”) and are involved in NIMR activity. Such NIMR family-binding polypeptides are also likely to be involved in the propagation of signals by the NIMR polypeptides or to associate with NIMR polypeptides and enhance or inhibit their activity.

The two-hybrid system is based on the modular nature of most transcription factors, which consist of separable DNA-binding and activation domains. Briefly, the assay utilizes two different DNA constructs. In one construct, the gene that codes for an NIMR polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NIMR polypeptide-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the NIMR polypeptide.

NIMR binding polypeptides may also be identified in other ways. For example, a library of molecules can be tested for the presence of NIMR binding polypeptides. In one embodiment, the library of molecules can be tested by expressing them in an expression vector, e.g., a bacteriophage. Bacteriophage can be made to display on their surface a plurality of polypeptide sequences, each polypeptide sequence being encoded by a nucleic acid contained within the bacteriophage. The phage expressing these candidate NIMR binding polypeptides can be tested for the ability to bind an immobilized NIMR polypeptide, to obtain those polypeptides having affinity for the NIMR polypeptide. For example, the method can comprise: contacting the immobilized NIMR polypeptide with a sample of the library of bacteriophage so that the NIMR polypeptide can interact with the different polypeptide sequences and bind those having affinity for the NIMR polypeptide to form a set of complexes consisting of immobilized NIMR polypeptide and bound bacteriophage. The complexes which have not formed a complex can be separated. The complexes of NIMR polypeptide and bound bacteriophage can be contacted with an agent that dissociates the bound bacteriophage from the complexes; and the dissociated bacteriophage can be isolated and the sequence of the nucleic acid molecule encoding the displayed polypeptide obtained, so that amino acid sequences of displayed polypeptides with affinity for NIMR polypeptides are obtained.

In the case of NIMR nucleic acid molecules, NIMR binding polypeptides can be identified, e.g., by contacting an NIMR nucleotide sequence with candidate NIMR binding polypeptides (e.g., in the form of microbial extract) under conditions which allow interaction of components of the extract with the NIMR nucleotide sequence. The ability of the NIMR nucleotide sequence to interact with the components can then be measured to thereby identify a polypeptide that binds to an NIMR nucleotide sequence.

2. Cell-Free Assays

The subject screening methods can involve cell-free assays, e.g., using high-throughput techniques. For example, to screen for agonists or antagonists, a synthetic reaction mix comprising an NIMR molecule and a labeled substrate or ligand of such polypeptide is incubated in the absence or the presence of a candidate molecule that may be an agonist or antagonist. In one embodiment, the reaction mix can further comprise a cellular compartment, such as a membrane, cell envelope or cell wall, or a combination thereof. The ability of the test compound to agonize or antagonize the NIMR polypeptide is reflected in decreased binding of the NIMR polypeptide to an NIMR binding polypeptide or in a decrease in NIMR polypeptide activity.

In many drug screening programs which test libraries of modulating agents and natural extracts, high throughput assays are desirable in order to maximize the number of modulating agents surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system.

In one embodiment, the ability of a compound to modulate the activity of an NIMR polypeptide is accomplished using isolated NIMR polypeptides or NIMR nucleic acid molecule in a cell-free system. In such an assay, the step of measuring the ability of a compound to modulate the activity of the NIMR polypeptide is accomplished, for example, by measuring direct binding of the compound to an NIMR polypeptide or NIMR nucleic acid molecule or the ability of the compound to alter the ability of the NIMR polypeptide to bind to a molecule to which the NIMR polypeptide normally binds (e.g., protein or DNA).

In yet another embodiment, an assay of the present invention is a cell-free assay in which an NIMR polypeptide or portion thereof is contacted with a test compound and the ability of the test compound to bind to the NIMR polypeptide or biologically active portion thereof is determined. Determining the ability of the test compound to modulate the activity of an NIMR polypeptide can be accomplished, for example, by determining the ability of the NIMR polypeptide to bind to an NIMR target molecule by one of the methods described above for determining direct binding. Determining the ability of the NIMR polypeptide to bind to an NIMR target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting an NIMR polypeptide or biologically active portion thereof with a known compound which binds the NIMR polypeptide to form an assay mixture, contacting the assay mixture with a test compound, and determining the ability of the test compound to interact with the NIMR polypeptide, wherein determining the ability of the test compound to interact with the NIMR polypeptide comprises determining the ability of the NIMR polypeptide to preferentially bind to or modulate the activity of an NIMR target molecule.

The cell-free assays of the present invention are amenable to use of both soluble and/or membrane-bound forms of proteins (e.g., NIMR polypeptides or NIMR binding polypeptides). In the case of cell-free assays in which a membrane-bound form of a polypeptide is used it may be desirable to utilize a solubilizing agent such that the membrane-bound form of the polypeptide is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

For example, compounds can be tested for their ability to directly bind to an NIMR nucleic acid molecule or an NIMR polypeptide or portion thereof, e.g., by using labeled compounds, e.g., radioactively labeled compounds. For example, an NIMR polypeptide sequence can be expressed by a bacteriophage. In this embodiment, phage which display the NIMR polypeptide would then be contacted with a compound so that the polypeptide can interact with and potentially form a complex with the compound. Phage which have formed complexes with compounds can then be separated from those which have not. The complex of the polypeptide and compound can then be contacted with an agent that dissociates the bacteriophage from the compound. Any compounds that bound to the polypeptide can then be isolated and identified.

In another embodiment, the ability of a compound to bind to an NIMR nucleic acid molecule can be measured. For example, gel shift assays or restriction enzyme protection assays can be used. Gel shift assays separate polypeptide-DNA complexes from free DNA by non-denaturing polyacrylamide gel electrophoresis. In such an experiment, the level of binding of a compound to DNA can be determined and compared to that in the absence of compound. Compounds which change the level of this binding are selected in the screen as modulating the activity of an NIMR polypeptide.

Other methods of assaying the ability of proteins to bind to DNA, e.g., DNA footprinting, and nuclease protection are also well known in the art and can be used to test the ability of a compound to bind to an NIMR nucleotide sequence.

In another embodiment, the invention provides a method for identifying compounds that modulate antibiotic resistance by assaying for test compounds that bind to NIMR nucleic acid molecules and interfere, e.g., with gene transcription.

In another embodiment, an NIMR nucleic acid molecule and an NIMR binding polypeptide that normally binds to that nucleotide sequence are contacted with a test compound to identify compounds that block the interaction of an NIMR nucleic acid molecule and an NIMR binding polypeptide. For example, in one embodiment, the NIMR nucleotide sequence and/or the NIMR binding polypeptide are contacted under conditions which allow interaction of the compound with at least one of the NIMR nucleic acid molecule and the NIMR binding polypeptide. The ability of the compound to modulate the interaction of the NIMR nucleotide sequence with the NIMR binding polypeptide is indicative of its ability to modulate an NIMR polypeptide activity.

Determining the ability of the NIMR polypeptide to bind to or interact with an NIMR binding polypeptide can be accomplished, e.g., by direct binding. In a direct binding assay, the NIMR polypeptide could be coupled with a radioisotope or enzymatic label such that binding of the NIMR polypeptide to an NIMR polypeptide target molecule can be determined by detecting the labeled NIMR polypeptide in a complex. For example NIMR polypeptides can be labeled with ¹²⁵I, ³⁵S, ¹⁴C, or ³H, either directly or indirectly, and the radioisotope detected by direct counting of radioemmission or by scintillation counting. Alternatively, NIMR polypeptide molecules can be enzymatically labeled with, for example, horseradish peroxidase, alkaline phosphatase, or luciferase, and the enzymatic label detected by determination of conversion of an appropriate substrate to product.

Typically, it will be desirable to immobilize either NIMR polypeptide, an NIMR binding polypeptide or a compound to facilitate separation of complexes from uncomplexed forms, as well as to accommodate automation of the assay. Binding of NIMR polypeptide to an upstream or downstream binding polypeptide, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the polypeptide to be bound to a matrix. For example, glutathione-S-transferase/NIMR polypeptide (GST/NIMR polypeptide) fusion proteins can be adsorbed onto glutathione sepharose beads (Sigma Chemical, St. Louis, Mo.) or glutathione derivatized microtitre plates, which are then combined with the cell lysates, e.g. an ³⁵S-labeled, and the test modulating agent, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of NIMR polypeptide-binding polypeptide found in the bead fraction quantitated from the gel using standard electrophoretic techniques.

Other techniques for immobilizing proteins on matrices are also available for use in the subject assay. For instance, either an NIMR polypeptide or polypeptide to which it binds can be immobilized utilizing conjugation of biotin and streptavidin. For instance, biotinylated NIMR polypeptide molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with NIMR polypeptide but which do not interfere with binding of upstream or downstream elements can be derivatized to the wells of the plate, and NIMR polypeptide trapped in the wells by antibody conjugation. As above, preparations of an NIMR polypeptide-binding polypeptide and a test modulating agent are incubated in the NIMR polypeptide-presenting wells of the plate, and the amount of complex trapped in the well can be quantitated. Exemplary methods for detecting such complexes, in addition to those described above for the GST-immobilized complexes, include immunodetection of complexes using antibodies reactive with the NIMR binding polypeptide, or which are reactive with NIMR polypeptide and compete with the binding polypeptide; as well as enzyme-linked assays which rely on detecting an enzymatic activity associated with the binding polypeptide, either intrinsic or extrinsic activity. In the instance of the latter, the enzyme can be chemically conjugated or provided as a fusion protein with the NIMR binding polypeptide. To illustrate, the NIMR polypeptide can be chemically cross-linked or genetically fused with horseradish peroxidase, and the amount of protein trapped in the complex can be assessed with a chromogenic substrate of the enzyme, e.g. 3,3′-diamino-benzadine terahydrochloride or 4-chloro-1-napthol. Likewise, a fusion protein comprising the protein and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the polypeptide, such as anti-NIMR polypeptide antibodies, can be used. Alternatively, the polypeptide to be detected in the complex can be “epitope tagged” in the form of a fusion protein which includes, in addition to the NIMR polypeptide sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, N.J.).

It is also within the scope of this invention to determine the ability of a compound to modulate the interaction between NIMR polypeptide and its target molecule, without the labeling of any of the interactants. For example, a microphysiometer can be used to detect the interaction of NIMR polypeptide with its target molecule without the labeling of either NIMR polypeptide or the target molecule. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

This invention further pertains to novel agents identified by the above-described screening assays. Accordingly, it is within the scope of this invention to further use an agent identified as described herein in methods of reducing drug resistance in microbes, e.g., in vivo or ex vivo. For example, an agent identified as described herein (e.g., an NIMR modulating agent such as an antisense NIMR nucleic acid molecule, an NIMR agonist or antagonist, or an NIMR-specific antibody) can be used in an animal model to determine the efficacy, toxicity, or side effects of treatment with such an agent. Alternatively, an agent identified as described herein can be used in an animal model to determine the mechanism of action of such an agent. Additionally, such agents can be used in methods of treatment (in vivo or ex vivo) or in methods of reducing resistance to drugs in the environment. Furthermore, this invention pertains to uses of novel agents identified by the above-described screening assays for treatments as described herein.

C. Vaccines

Another aspect of the invention relates to a method for inducing an immunological response in an individual, particularly a mammal, comprising inoculating the individual with an NIMR modulating agent, or a fragment or variant thereof, adequate to produce an immune response and/or to augment an immune response (e.g., an antibody and/or T cell immune response) to ameliorate or prevent infection with a microbe comprising an NIMR polypeptide. The invention also relates to a method of inducing immunological response in an individual which comprises delivering to such individual a nucleic acid vector to direct expression of an NIMR molecule, or a fragment or a variant thereof, for expressing an NIMR molecule, or a fragment or a variant thereof in vivo in order to induce an immunological response, such as, to produce antibody and/or T cell immune response, including, for example, cytokine-producing T cells or cytotoxic T cells, to ameliorate an ongoing infection or to prevent infection. One way of administering the gene is by accelerating it into the desired cells as a coating on particles or otherwise. Such nucleic acid vector may comprise, e.g., DNA, RNA, a modified nucleic acid, or a DNA/RNA hybrid.

A further aspect of the invention relates to an immunological composition which, when introduced into an individual, induces an immunological response. Such a composition can comprise, e.g., an isolated NIMR polypeptide or an NIMR nucleic acid molecule. The immunologic compsition may be used therapeutically or prophylactically and may be dominated by either a humoral response or a cellular immune response.

In one embodiment, an NIMR polypeptide or a fragment thereof may be fused with a second polypeptide, which may not by itself produce antibodies, but is capable of stabilizing the first polypeptide and enhancing immunogenic and protective properties. Thus fused recombinant polypeptide, preferably further comprises an antigenic co-protein, such as lipoprotein D from Hemophilus influenzae, Glutathione-S-transferase (GST) or beta-galactosidase, relatively large second proteins which solubilize the polypeptide and facilitate production and purification of an NIMR molecule to which they are fused. Moreover, the second polypeptide may act as an adjuvant in the sense of providing a generalized stimulation of the immune system. The second polypeptide may be attached to either the amino or carboxy terminus of the NIMR polypeptide.

The use of a nucleic acid molecule of the invention in genetic immunization will preferably employ a suitable delivery method such as direct injection of plasmid DNA into muscles (Wolff et al., Hum Mol Genet 1992, 1:363, Manthorpe et al., Hum. Gene Ther. 1963:4, 419), delivery of DNA complexed with specific polypeptide carriers (Wu et al., J. Biol. Chem. 1989: 264, 16985), coprecipitation of DNA with calcium phosphate (Benvenisty & Reshef, PNAS USA, 1986:83, 9551), encapsulation of DNA in various forms of liposomes (Kaneda et al., Science 1989:243, 375), particle bombardment (Tang et al., Nature 1992, 356:152, Eisenbraun et al., DNA Cell Biol 1993, 12:791) and in vivo infection using cloned retroviral vectors (Seeger et al., PNAS USA 1984:81, 5849).

In one embodiment, immunostimulatory DNA sequences, such as those described in Sato, Y. et al. Science 273: 352 (1996) can be used in connection with the instant invention.

In one embodiment, a vaccine formulation comprises an immunogenic recombinant polypeptide of the invention together with a suitable carrier. Preferably, such vaccines are administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostats and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems, alum, or other systems known in the art. The dosage will depend on the specific activity of the vaccine and on the status of the patient and can be readily determined by routine experimentation.

VI. Compositions Comprising NIMR Modulating Agents

The compositions of the invention can comprise at least one NIMR modulating agent and one or more pharmaceutically acceptable carriers (additives) and/or diluents. A composition can also include a second antimicrobial agent, e.g., an antimicrobial compound, preferably an antibiotic or a non-antibiotic agent.

As described in detail below, the compositions can be formulated for administration in solid or liquid form, including those adapted for the following: (George, A. M. & Levy, S. B. (1983) J. Bacteriol. 155, 541-548) oral administration, for example, drenches (aqueous or non-aqueous solutions or suspensions), tablets, boluses, powders, granules, pastes; (Cohen, S. P., Yan, W. & Levy, S. B. (1993) J. Infect. Dis. 168, 484-488) parenteral administration, for example, by subcutaneous, intramuscular or intravenous injection as, for example, a sterile solution or suspension; (Cohen, S. P., Hachler, H. & Levy, S. B. (1993) J. Bacteriol. 175, 1484-1492) topical application, for example, as a cream, ointment or spray applied to the skin; (Sulavick, M. C.; Dazer, M. & Miller, P. F. (1997) J. Bacteriol. 179, 1857-1866) intravaginally or intrarectally, for example, as a pessary, cream, foam, or suppository; or (Cohen, S. P., Levy, S. B., Foulds, J. & Rosner, J. L. (1993) J. Bacteriol 175, 7856-7862) aerosol, for example, as an aqueous aerosol, liposomal preparation or solid particles containing the compound.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the antimicrobial agents or compounds of the invention from one organ, or portion of the body, to another organ, or portion of the body without affecting its biological effect. Each carrier should be “acceptable” in the sense of being compatible with the other ingredients of the composition and not injurious to the subject. Some examples of materials which can serve as pharmaceutically-acceptable carriers include: (George, A. M. & Levy, S. B. (1983) J. Bacteriol. 155, 541-548) sugars, such as lactose, glucose and sucrose; (Cohen, S. P., Yan, W. & Levy, S. B. (1993) J. Infect. Dis. 168, 484-488) starches, such as corn starch and potato starch; (Cohen, S. P., Hachler, H. & Levy, S. B. (1993) J. Bacteriol. 175, 1484-1492) cellulose, and its derivatives, such as sodium carboxymethyl cellulose, ethyl cellulose and cellulose acetate; (Sulavick, M. C., Dazer, M. & Miller, P. F. (1997) J. Bacteriol. 179, 1857-1866) powdered tragacanth; (Cohen, S. P., Levy, S. B., Foulds, J. & Rosner, J. L. (1993) J. Bacteriol 175, 7856-7862) malt; (Alekshun, M. A. & Levy, S. B. (1999) J. Bacteriol. 181, 4669-4672) gelatin; (George, A. M. & Levy, S. B. (1983) J. Bacteriol. 155, 531-540) talc; (Oethinger, M., Podglajen, I., Kern, W. V. & Levy, S. B. (1998) Antimicrob. Agents Chemother. 42, 2089-2094) excipients, such as cocoa butter and suppository waxes; (Asako, H., Nakajima, K., Kobayashi, K., Kobayashi, M. & Aono, R. (1997) Appl. Environ. Microbiol 63, 1428-1433) oils, such as peanut oil, cottonseed oil, safflower oil, sesame oil, olive oil, corn oil and soybean oil; (White, D. G., Goldman, J. D., Demple, B. & Levy, S. B. (1997) J. Bacteriol. 179, 6122-6126) glycols, such as propylene glycol; (Ariza, R. R., Cohen, S. P., Bachbawat, N., Levy, S. B. & Demple, B. (1994) J. Bacteriol. 176, 143-148) polyols, such as glycerin, sorbitol, mannitol and polyethylene glycol; (McMurry, L. M., Oethinger, M. & Levy, S. B. (1998) FEMS Microbiol. Lett. 166, 305-309) esters, such as ethyl oleate and ethyl laurate; (Moken, M. C., McMurry, L. M. & Levy, S. B. (1997) Antimiclob. Agents Chemother. 41, 2770-2772) agar; (Martin, R. G., Gillette, W. K., Rhee, S. & Rosner, J. L. (1999) Mol. Microbiol. 34, 431-441) buffering agents, such as magnesium hydroxide and aluminum hydroxide; (Maneewannakul, K. & Levy, S. B. (1996) Antimicrob. Agents Chemother. 40, 1695-1698) alginic acid; (Seoane, A. S. & Levy, S. B. (1995) J. Bacteriol. 177, 530-535) pyrogen-free water; (Hamilton, C. M., Aldea, M., Washburn, B. K., Babitzke, P. & Kushner, S. R. (1989) J. Bacteriol. 171, 4617-4622) isotonic saline; (Sambrook, J., Fritsch, E. F. & Maniatis, T. (1989) in Molecular Cloning. A Laboratory Manual, eds. Cold Spring Harbor Laboratory Press (Cold Spring Harbor, N.Y.)) Ringer's solution; (Blattner, F. R., Plunkett, G. I. I. I., Bloch, C. A., Perna, N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462) ethyl alcohol; (Tao, H., Bausch, C., Richmond, C., Blattner, F. R. & Conway, T. (1999) J. Bacteriol. 181, 6425-6440) phosphate buffer solutions; and (Alekshun, M. N. & Levy, S. B. (1997) Antimicrob. Agents Chemother. 41, 2067-2075) other non-toxic compatible substances employed in pharmaceutical compositions. Proper fluidity can be maintained, for example, by the use of coating materials, such as lecithin, by the maintenance of the required particle size in the case of dispersions, and by the use of surfactants.

These compositions may also contain additional agents, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Compositions of the present invention may be administered to epithelial surfaces of the body orally, parenterally, topically, rectally, nasally, intravaginally, intracisternally. They are of course given by forms suitable for each administration route. For example, they are administered in tablets or capsule form, by injection, inhalation, eye lotion, ointment, etc., administration by injection, infusion or inhalation; topical by lotion or ointment; and rectal or vaginal suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a sucrose octasulfate and/or an antibacterial or a contraceptive agent, drug or other material other than directly into the central nervous system, such that it enters the subject's system and, thus, is subject to metabolism and other like processes, for example, subcutaneous administration.

In some methods, the compositions of the invention can be topically administered to any epithelial surface. An “epithelial surface” according to this invention is defined as an area of tissue that covers external surfaces of a body, or which and lines hollow structures including, but not limited to, cutaneous and mucosal surfaces. Such epithelial surfaces include oral, pharyngeal, esophageal, pulmonary, ocular, aural, nasal, buccal, lingual, vaginal, cervical, genitourinary, alimentary, and anorectal surfaces.

Compositions can be formulated in a variety of conventional forms employed for topical administration. These include, for example, semi-solid and liquid dosage forms, such as liquid solutions or suspensions, suppositories, douches, enemas, gels, creams, emulsions, lotions, slurries, powders, sprays, lipsticks, foams, pastes, toothpastes, ointments, salves, balms, douches, drops, troches, chewing gums, lozenges, mouthwashes, rinses.

Conventionally used carriers for topical applications include pectin, gelatin and derivatives thereof, polylactic acid or polyglycolic acid polymers or copolymers thereof, cellulose derivatives such as methyl cellulose, carboxymethyl cellulose, or oxidized cellulose, guar gum, acacia gum, karaya gum, tragacanth gum, bentonite, agar, carbomer, bladderwrack, ceratonia, dextran and derivatives thereof, ghatti gum, hectorite, ispaghula husk, polyvinypyrrolidone, silica and derivatives thereof, xanthan gum, kaolin, talc, starch and derivatives thereof, paraf fin, water, vegetable and animal oils, polyethylene, polyethylene oxide, polyethylene glycol, polypropylene glycol, glycerol, ethanol, propanol, propylene glycol (glycols, alcohols), fixed oils, sodium, potassium, aluminum, magnesium or calcium salts (such as chloride, carbonate, bicarbonate, citrate, gluconate, lactate, acetate, gluceptate or tartrate).

Such compositions can be particularly useful, for example, for treatment or prevention of an unwanted infections e.g., of the oral cavity, including cold sores, infections of eye, the skin, or the lower intestinal tract. Standard composition strategies for topical agents can be applied to the antimicrobial compounds, or pharmaceutically acceptable salts thereof in order to enhance the persistence and residence time of the drug, and to improve the prophylactic efficacy achieved.

For topical application to be used in the lower intestinal tract or vaginally, a rectal suppository, a suitable enema, a gel, an ointment, a solution, a suspension or an insert can be used. Topical transdermal patches may also be used. Transdermal patches have the added advantage of providing controlled delivery of the compositions of the invention to the body. Such dosage forms can be made by dissolving or dispersing the agent in the proper medium.

Compositions of the invention can be administered in the form of suppositories for rectal or vaginal administration. These can be prepared by mixing the agent with a suitable non-irritating carrier which is solid at room temperature but liquid at rectal temperature and therefore will melt in the rectum or vagina to release the drug. Such materials include cocoa butter, beeswax, polyethylene glycols, a suppository wax or a salicylate, and which is solid at room temperature, but liquid at body temperature and, therefore, will melt in the rectum or vaginal cavity and release the active agent.

Compositions which are suitable for vaginal administration also include pessaries, tampons, creams, gels, pastes, foams, films, or spray compositions containing such carriers as are known in the art to be appropriate. The carrier employed in the sucrose octasulfate/contraceptive agent should be compatible with vaginal administration and/or coating of contraceptive devices. Combinations can be in solid, semi-solid and liquid dosage forms, such as diaphragm, jelly, douches, foams, films, ointments, creams, balms, gels, salves, pastes, slurries, vaginal suppositories, sexual lubricants, and coatings for devices, such as condoms, contraceptive sponges, cervical caps and diaphragms.

For ophthalmic applications, the pharmaceutical compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions can be formulated in an ointment such as petrolium. Exemplary ophthalmic compositions include eye ointments, powders, solutions and the like.

Powders and sprays can contain, in addition to sucrose octasulfate and/or antibiotic or contraceptive agent(s), carriers such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Compositions of the invention can also be orally administered in any orally-acceptable dosage form including, but not limited to, capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of sucrose octasulfate and/or antibiotic or contraceptive agent(s) as an active ingredient. A compound may also be administered as a bolus, electuary or paste. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Tablets, and other solid dosage forms, such as dragees, capsules, pills and granules, may be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the antimicrobial agent(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Sterile injectable forms of the compositions of this invention can be aqueous or oleaginous suspensions. These suspensions may be formulated according to techniques known in the art using suitable dispersing or wetting agents and suspending agents. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The sterile injectable preparation may also be a sterile injectable solution or suspension in a nontoxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

In the case of modulators of the activity and/or expression of NIMR molecules which are nucleic acid molecules, the optimal course of administration of the oligomers may vary depending upon the desired result or on the subject to be treated. As used in this context “administration” refers to contacting cells with oligomers, e.g., in vivo or ex vivo. The dosage of nucleic molecule may be adjusted to optimally regulate expression of a protein translated from a target mRNA, e.g., as measured by a readout of RNA stability or by a therapeutic response, without undue experimentation. For example, expression of the protein encoded by the nucleic acid can be measured to determine whether or dosage regimen needs to be adjusted accordingly. In addition, an increase or decrease in RNA and/or protein levels in a cell or produced by a cell can be measured using any art recognized technique. By determining whether transcription has been decreased, the effectiveness of the molecule can be determined.

As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

Compositions may be incorporated into liposomes or liposomes modified with polyethylene glycol or admixed with cationic lipids for parenteral administration. Incorporation of additional substances into the liposome, for example, antibodies reactive against membrane proteins found on specific target microbes, can help target the molecule to specific cell types.

Moreover, the present invention provides for administering the subject compositions with an osmotic pump providing continuous infusion of the compositions, for example, as described in Rataiczak et al. (1992 Proc. Natl. Acad. Sci. USA 89:11823-11827). Such osmotic pumps are commercially available, e.g., from Alzet Inc. (Palo Alto, Calif.). Topical administration and parenteral administration in a cationic lipid carrier are preferred.

With respect to in vivo applications, the formulations of the present invention can be administered to a patient in a variety of forms adapted to the chosen route of administration, namely, parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation. Intravenous administration is preferred among the routes of parenteral administration.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran, optionally, the suspension may also contain stabilizers.

Drug delivery vehicles can be chosen e.g., for in vitro, for systemic, or for topical administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The subject compositions may be incorporated into liposomes or liposomes modified with polyethylene glycol or admixed with cationic lipids for parenteral administration. Incorporation of additional substances into the liposome, for example, antibodies reactive against membrane proteins found on specific target microbes, can help target the compositions to specific cell types.

Moreover, the present invention provides for administering the subject compositions with an osmotic pump providing continuous infusion of nucleic acid molecules, for example, as described in Rataiczak et al. (1992 Proc. Natl. Acad. Sci. USA 89:11823-11827). Such osmotic pumps are commercially available, e.g., from Alzet Inc. (Palo Alto, Calif). Topical adminis tration and parenteral administration in a cationic lipid carrier are preferred.

With respect to in vivo applications, the formulations of the present invention can be administered to a patient in a variety of forms adapted to the chosen route of administration, namely, parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation. Intravenous administration is preferred among the routes of parenteral administration.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran, optionally, the suspension may also contain stabilizers.

Drug delivery vehicles can be chosen e.g., for in vitro, for systemic, or for topical administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The described compositions may be administered systemically to a subject. Systemic absorption refers to the entry of drugs into the blood stream followed by distribution throughout the entire body. Administration routes which lead to systemic absorption include: intravenous, subcutaneous, intraperitoneal, and intranasal. Each of these administration routes delivers the compositions to accessible diseased cells. Following subcutaneous administration, the therapeutic agent drains into local lymph nodes and proceeds through the lymphatic network into the circulation. The rate of entry into the circulation has been shown to be a function of molecular weight or size. The use of a liposome or other drug carrier localizes the compositions at the lymph node. The nucleic acid molecule can be modified to diffuse into the cell, or the liposome can directly participate in the delivery of the composition into the cell.

For prophylactic applications, the pharmaceutical composition of the invention can be applied prior to physical contact with a microbe. The timing of application prior to physical contact can be optimized to maximize the prophylactic effectiveness of the compound. The timing of application will vary depending on the mode of administration, the epithelial surface to which it is applied, the surface area, doses, the stability and effectiveness of composition under the pH of the epithelial surface, the frequency of application, e.g., single application or multiple applications. Preferably, the timing of application can be determined such that a single application of composition is sufficient. One skilled in the art will be able to determine the most appropriate time interval required to maximize prophylactic effectiveness of the compound.

One of ordinary skill in the art can determine and prescribe the effective amount of the pharmaceutical composition required. For example, one could start doses at levels lower than that required in order to achieve the desired therapeutic effect and gradually increase the dosage until the desired effect is achieved. In general, a suitable daily dose of a composition of the invention will be that amount of the composition which is the lowest dose effective to produce a therapeutic effect. Such an effective dose will generally depend upon the factors described above. It is preferred that administration be intravenous, intracoronary, intramuscular, intraperitoneal, or subcutaneous.

Another aspect of the invention pertains to kits for carrying out the screening assays or modulatory methods of the invention. For example, a kit for carrying out a screening assay of the invention can include a cell comprising an NIMR polypeptide, means for determining NIMR polypeptide activity and instructions for using the kit to identify modulators of NIMR activity.

In another embodiment, the invention provides a kit for carrying out a modulatory method of the invention. The kit can include, for example, a modulatory agent of the invention (e.g., an NIMR inhibitory or stimulatory agent) in a suitable carrier and packaged in a suitable container with instructions for use of the modulatory agent to modulate NIMR expression or activity.

The practice of the present invention will employ, unless otherwise indicated, conventional techniques of cell biology, cell culture, molecular biology, genetics, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et. al. (Cold Spring Harbor Laboratory Press (1989)); Short Protocols in Molecular Biology, 3rd Ed., ed. by Ausubel, F. et al. (Wiley, NY (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The contents of all references, pending patent applications and published patents, cited throughout this application are hereby expressly incorporated by reference. Each reference disclosed herein is incorporated by reference herein in its entirety. Any patent application to which this application claims priority is also incorporated by reference herein in its entirety.

The invention is further illustrated by the following examples, which should not be construed as further limiting.

### EXAMPLES

### Example 1

The following materials and methods were used in the examples:

Bacterial strains, plasmids and growth conditions. E. coli K-12 strain AG100 (George, A. M. & Levy, S. B. (1983) J. Bacteriol. 155, 541-548) was used for the PCR amplification of specific DNA probes. E coli AG100Kan, an isogenic strain of AG100 containing a 1.2 kb kanamycin resistance cassette in the place of the mar locus (Maneewannakul, K. & Levy, S. B. (1996) Antimicrob. Agents Chemother. 40, 1695-1698) was used in all the experiments described. pAS10 (Seoane, A. S. & Levy, S. B. (1995) J. Bacteriol. 177, 530-535), derived from the temperature-sensitive pMAK705 (Chl^{R}) (Hamilton, C. M., Aldea, M., Washburn, B. K., Babitzke, P. & Kushner, S. R. (1989) J. Bacteriol. 171, 4617-4622), carries a 2.5 kb PCR amplified fragment containing the marCORAB sequence bearing the marR5 mutation, which produces no MarR and thus mediates constitutive MarA expression.

Bacterial strains were grown in Luria Bertani (LB) media (composition per litre: 10 g tryptone, 10 g NaCl, 5 g yeast extract) at 30° C. with vigorous aeration. E. coli AG100Kan cells were made competent by the standard CaCl₂, method (Sambrook, J., Fritsch, E. F. & Maniatis, T. (1989) in Molecular Cloning. A Laboratory Manual, eds. Cold Spring Harbor Laboratory Press (Cold Spring Harbor, N.Y.)) and transformants containing the plasmids pMAK705 or pAS10 were maintained in the presence of 25 μg ml⁻¹ chloramphenicol (Sigma, St. Louis, Mo.).

RNA extraction. Total RNA was isolated by a modification of the hot-acidic phenol extraction method (Sigma-Genosys Biotechnologies, Inc., The Woodlands, Tex.). Overnight cultures were diluted 250-fold in fresh LB medium, and grown to mid-logarithmic phase (A₅₃₀=0.35-0.40). Bacterial pellets from 5 ml cell cultures were harvested at 4° C., and resuspended in 250 μl ice-cold resuspension buffer (0.3 M sucrose-10 mM sodium acetate, pH 4.2) and 37.5 μl of ice-cold 0.5 M EDTA. After incubation on ice for 5 min, cells were lysed by adding 375 μl lysis buffer (2%, sodium dodecyl sulphate, 10 mM sodium acetate, pH 4.2) and heating at 65° C. for 3 min. The suspension was extracted three times with 700 μl of pre-warmed acidic phenol (65° C.) (Sigma) and the aqueous phase was extracted, first with 700 μl of a mixture of acidic phenol:chlorophorm:isoamyl alcohol (25:24:1), and then with an equal volume of chlorophorm:isoamylalcollol (24:1). The RNA in the aqueous phase was ethanol precipitated at −80° C., and the RNA pellet rinsed with 70% ethanol and resuspended in 100 μl of RNase-free water (Ambion Inc., Austin, Tex.). Samples were treated with DNaseI (amplification grade, Life Technologies Inc., Gaithersburg, Md.), following the manufacturer's instructions, to eliminate DNA contamination. The absence of genomic DNA in the RNA was confirmed by examining samples of the RNA in non-denaturing 1.2%, agarose gels, and by performing PCR on DNase treated RNA samples using primers known to target the genomic DNA. The RNA concentration was determined spectrophotometrically (Sambrook, J., Fritsch, E. F. & Maniatis, T. (1989) in Molecular Cloning A Laboratory Manual, eds. Cold Spring Harbor Laboratory Press (Cold Spring Harbor, N.Y.)).

Preparation of labeled cDNA and hybridization to the arrays. Labeled cDNA was prepared using the E. coli cDNA labeling primers (Sigma-Genosys) following the manufacturer's instructions. The primers were annealed to 1 μg of total RNA in the presence of 333 μM dATP, dCTP and dTTP and 1× reverse transcriptase buffer at 90° C. for 2 min. The mixture was cooled to 42° C. and 50 U AMV reverse transcriptase (Boehringer-Mannheim, Indianapolis, Ind.) and 20 μCi ³²P-α-dTP (2,000 Ci/mmol) (New England Nuclear, Boston, Mass.) were added. Incubation was at 42° C. for 2 h 30 min. The unincorporated nucleotides were removed from the labeled cDNA using a NucTrap probe purification column (Stratagene, La Jolla, Calif.) prior to hybridization. Hybridization of the purified labeled cDNA to the Panorama E. coli Gene arrays (SigmaGenosys) was performed in roller bottles following the manufacturer's instructions. Essentially, arrays were pre-wet in 2×SSPE and then pre-hybridized for 2 h at 65° C. in 5 ml pre-warned hybridization solution (5×SSPE, 2% SDS, 1× Denhardt's reagent and 100 μg ml⁻¹ denatured salmon sperm DNA). Denatured labeled cDNA in 5 ml hybridization solution replaced the prehybridization solution and hybridization proceeded for ˜18 h at 65° C. The arrays were washed 3× with 50 ml wash buffer (0.5×SSPE-0.2% SDS) at room temperature for 3 min intervals and 3× with 100 ml pre-warmed (65° C.) wash buffer for 20 min intervals. Hybridizing signals on the membrane were visualized by exposure to Kodak BioMax MR X-ray film and to a Kodak storage phosphorimager screen SO230 (Molecular Dynamics, Sunnivale, Calif.). Phosphor screens were scanned, after 1 to 3 days exposure, at 50 micron pixel resolution in a Storm 860 phosphorimaging instrument (Molecular Dynamics). Arrays were stripped by immersing the membranes in a boiling solution of 0.5% SDS (w/v) and removal of the probe was confirmed before reuse as described above.

Description and quantification of the arrays. The Panorama E. coli Gene Arrays (Sigma-Genosys) contain 4,290 PCR-amplified Orfs of the E. coli K-12 (MG1655) genome (Blattner, F. R., Plunkett, G. 1.1. I., Bloch, C. A., Perna, N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462), spotted in duplicate (see Tao et al. (Tao, H., Bausch, C., Richmond, C., Blattner, F. R. & Conway, T. (1999) J. Bacteriol. 181, 6425-6440) for a more detailed description of the arrays).

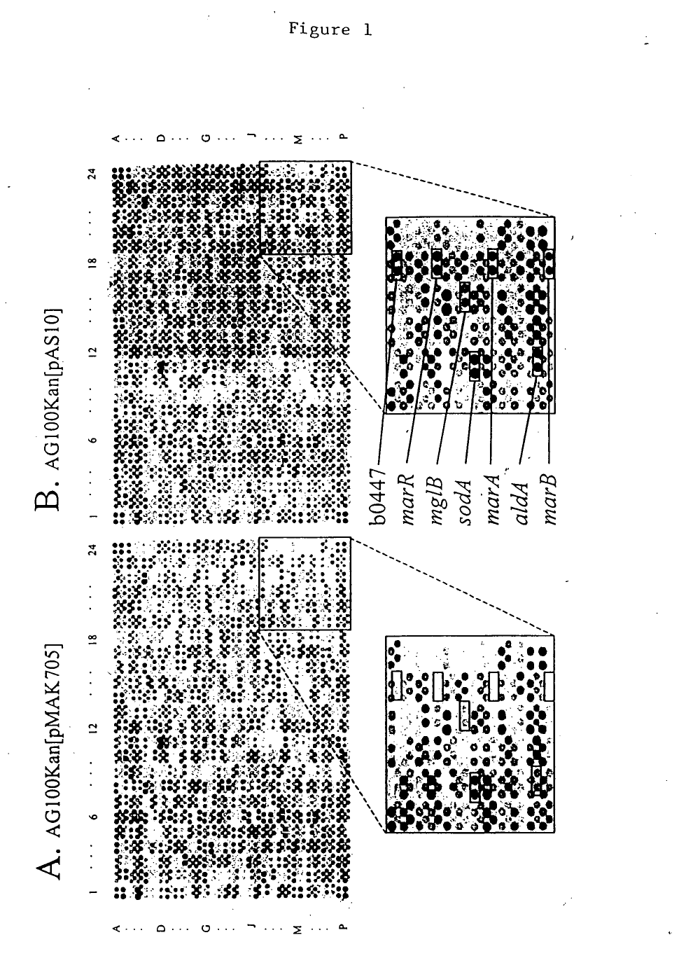

Quantification of the hybridizing signals in the phosphoimager file was carried out by Sigma-Genosys using the Array Vision&Trade software (Imaging Research, Inc.). The relative pixel values for the duplicate spots of each gene were averaged and normalized by expressing the averaged spot signal as a percentage to the signal from the averaged pixel values of the genomic DNA spots in the respective field where each gene was printed (FIG. 1). In FIG. 1, The ratio between these values in samples from cells expressing or lacking MarA represented the fold change in gene expression. Background values were determined for each field in each array by averaging the pixel values of the empty spaces located in the same secondary grid as the genomic DNA (FIG. 1). Genes whose averaged pixel values were close to background (less than a 2-fold difference from background values) in both experimental and control samples were not considered here. Identical arrays were probed with labeled ³²P-cDNA populations prepared from total RNA from mar-deleted, AG100Kan[pMAK705] (panel A) and mar-expressing, AG100Kan[pAS00] (panel B) strains. Columns (1-24) and rows (A-P) forming the primary grid in Field 1 of the autoradiogram are labeled. Fields 2 and 3 are similar in format to Field I and are not shown. The four spots in the four corners of each field are genomic DNA. Boxes underneath correspond to expanded views of representative areas shown in (A) and (B) where changes in expression levels are visible for several genes (7 of the differentially expressed genes are labeled as examples).

All the genes identified by computing analysis as members of the mar regulon were confirmed by visual analysis of autoradiograms of the arrays in three independent experiments. Only those genes which satisfied both criteria were classified as members of the mar regulon.

Northern blot analysis. Duplicate samples of DNaseI treated total RNA (5-10 μg) were fractionated electrophoretically on 1-1.2%, denaturing formaldehyde-agarose gels, and RNA was transferred to nylon membranes (Hybond-N, Amersham Life Science Inc., Arlington Heights, Ill.) using established capillary blotting methods in 10×SSC (Sambrook, J., Fritsch, E. F. & Maniatis, T. (1989) in Molecular Cloning. A Laboratory Manual, eds. Cold Spring Harbor Laboratory Press (Cold Spring Harbor, N.Y.).). DNA probes for specific E. coli genes were amplified by PCR from E. coli AG100 chromosomal DNA using the appropriate E. coli ORFmer PCR primer pairs (Sigma-Genosys), according to the supplier specifications. After amplification, the PCR products were purified from agarose gels using the Qiaex II gel extraction kit (Qiagen Inc., Valencia, Calif.) and quantified by comparison to DNA size standards (Life Technologies) of known concentration. Labeling of DNA probes with [³²P]-dCTP (New England Nuclear) using the RTS RadPrime DNA labeling system (Life Technologies) was carried out according to the manufacturer's instructions. Hybridizations were performed using standard procedures at 65° C. (Sambrook, J., Fritsch, E. F. & Maniatis, T. (1989) in Molecular Cloning. A Laboratory Manual, eds. Cold Spring Harbor Laboratory Press (Cold Spring Harbor, N.Y.).), and RNA membranes were washed at high stringency for 15 min intervals, four times in 2×SSC buffer/0.1% SDS and 2 to 4 times in 0.1×SSC buffer/0.1% SDS. Hybridizing bands were visualized as described for the E. coli gene macroarrays.

DNA manipulations. Genomic and plasmid DNA were purified from E. coli strains using the QIAamp Tissue kit and the QIAprep spin Miniprep kit (Qiagen) respectively, following manufacturer's instructions.

### EXAMPLE 1

### Identification of genes regulated by MarA. DNA macroarrays, constructed for E. coli, which contain most of the genomic Orfs (Blattner, F. R., Plunkett, G. I. I. I., Bloch, C. A., Perna, N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462), allowed studies of expression of the complete genome in the presence or absence of MarA. E. coli AG100K a strain (Maneewannakul, K. & Levy, S. B. (1996) Antimicrob. Agents Chemother. 40, 1695-1698) bearing only plasmid pMAK705 represented the control, i.e. deficient in mar expression. The experimental strain AG100Kan[pAS10] containing the pMAK705-derived plasmid pAS10, which expresses MarA constitutively (Seoane, A. S. & Levy, S. B. (1995) J. Bacteriol. 177, 530-535). Antibiotic susceptibility assayed using the E-test method showed the expected increase (˜4-20 fold) in resistance in the mar expressing strain as corn pared to the control to the antibiotics tested, including norfloxacin, nalidixic acid, tetracycline and ampicillin (data not shown).

³³P-labeled cDNAs prepared from RNA extracted from mar-deleted and mar-expressing strains were hybridized to paired macroarrays and phosphorimager files and autoradiograms were obtained (FIG. 1). Previously ˜15 genes were known to be regulated by MarA (Alekshun, M. N. & Levy, S. B. (1997) Antimicrob. Agents Chemother. 41, 2067-2075). The gene macroarrays identified a total of 62 genes responsive to mar-regulation in logarithmic phase: 47 induced and 15 repressed (Table 3). Only those findings detected in all three experiments were included in the list.

The signals for the three genes encoded by the marRAB operon were easily detected in the cDNA from the mar-expressing but not from the mar-deleted strain (FIG. 1). This finding was reassuring given that cDNAs from genes belonging to the same family of homologues (e.g. soxS and rob for marA) could have caused some level of non-specific binding (Richmond, C. S., Glasner, J. D., Mau, R., Jin, H. & Blattner, F. R. (1999) Nucleic Acids Res. 27, 38213835). For marR, marA and marB, the expression was 31-fold, 35-fold and 12-fold higher (averaged values) than in control samples (Table 3). Although the signal for marB expression was consistently less than the signals for marR and marA expression the meaning is unclear. Since the spotted PCR products differ in length (which has an effect in hybridizing intensities, (Richmond, C. S., Glasner, J. D., Mau, R., Jin, H. & Blattner, F. R. (1999) Nucleic Acids Res. 27, 38213835)), and because the efficiency of reverse transcription will vary between different RNAs, the results do not allow comparative analysis between different genes. The expression of the divergent marC, (referred to as ydeB in GenBank), was close to background in the experimental sample. Thus it does not appear to be affected by MarA under these conditions.

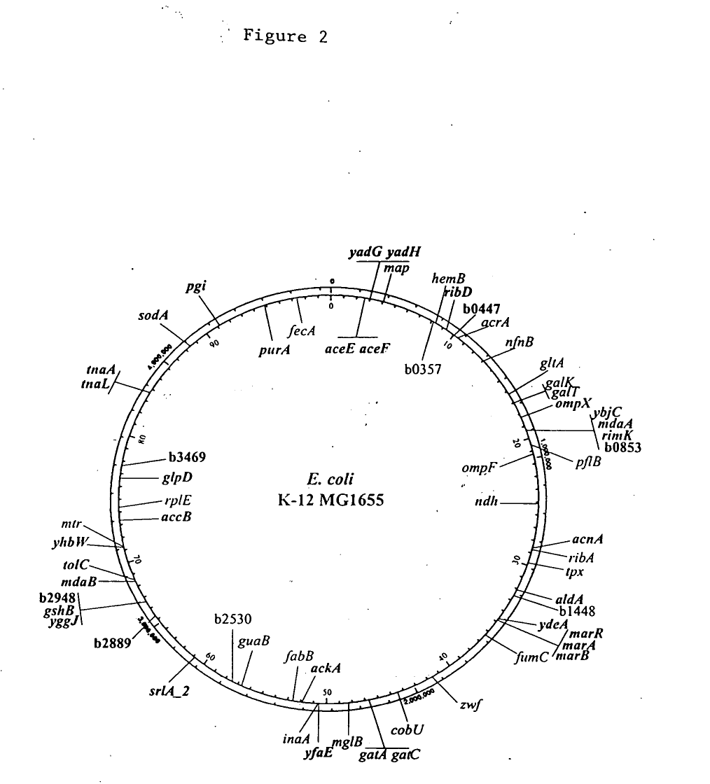

The mar-regulated genes identified are dispersed throughout the chromosome and are involved in a wide range of cell functions (FIG. 2, Table 2). In FIG. 2, the internal circle represents the chromosome of E. coli K-12 MG1655 divided in intervals of 1 minute, while the external is divided in intervals of 100,000 nucleotide residues (adapted from Blattner et al. (Blattner, F. R., Plunkett, G. I. I. I., Bloch, C. A., Perna, N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462)). Genes induced by mar are plotted to face the exterior of the chromosome and genes repressed by mar are plotted to face the interior of the chromosome. Bold faced genes read in the clockwise direction, while regular font represents those genes on the opposite strand (Blattner, F. R., Plunkett, G. I. I. I., Bloch, C. A., Perna; N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462). Genes that are in the immediate vicinity of each other were placed together over the same designation line.

In addition to changing the expression of genes with known functions, MarA also changed the expression of genes yet uncharacterized. For instance the gene bO447 encodes a putative LRP-like transcriptional regulator, yadG encodes a putative ATP-binding component of a transport system, while bl448 and yggJ have no known homologues. It is not clear how all these genes relate to each other in the development of the Mar phenotype. gshB is involved in the synthesis of glutathione, which is part of the cell's antioxidants defenses (Hidalgo, E. & Demple, B. (1995) in Regulation of gene expression in Escherichia coli., eds. Lin, E. C. & Lynch, A. S. (R. G. Landes Company, Austin), pp. 433-450), and among other functions, is involved in the reduction of OxyR to its normal redox state (Chater, K. F. & Nikaido, H. (1999) Curr. Opin. Microbiol. 2, 121-125) and in the detoxification of toxic electrophiles (Ferguson, G. P. (1999) Trends Microbiol. 7, 242-247). The induction of gshB by MarA could help to explain why resistance to oxidative stress is a Mar phenotype.

### Example 2

### Confirmation of Previously Identified mar Regulated Genes

The differential expression of most of the genes previously identified as part of the, mar regulon, e.g. inaA, sodA, ompF, zwf and fumC (Ariza, R. R., Cohen, S. P., Bachbawat, N., Levy, S. B. & Demple, B. (1994) J. Bacteriol. 176, 143-148, Greenberg, J. T., Chou, J. H., Monach, P. A. & Demple, B. (1991) J. Bacteriol. 173, 4433-4439, Jair, K.-W., Martin, R. G., Rosner, J. L., Fujita, N., Ishihama, A. & Wolf, J. R. E. (1995) J. Bacteriol 177, 7100-7104, Rosner, J. L. & Slonczewski, J. L. (1994) J. Bacteriol. 176, 6262-6269), was confirmed in the current study (Table 3). A major role in the Mar phenotype is played by the efflux system acrAB, which acts by pumping toxic compounds out of the cell (White, D. G., Goldman, J. D., Demple, B. & Levy, S. B. (1997) J. Bacteriol. 179, 6122-6126, Moken, M. C., McMurry, L. M. & Levy, S. B. (1997) Antimiclob. Agents Chemother. 41, 2770-2772, Okusu, H., Ma, D. & Nikaido, H. (1996) J. Bacteriol. 178, 306-308). An increase in the expression of the acrA gene of the acrAB operon was also observed (Table 3), however the expression values for acrB were not above background. As described earlier for marB, this kind of finding is not fully understood, but could arise from differential processing of the polycistronic transcript and/or by slight differences in transcript stability.

Previous studies suggest co-ordinate activation of TolC and the AcrAB efflux pump in the development of the Mar phenotype, particularly in the context of organic solvent tolerance (Fralick, J. A. (1996) J. Bacteriol. 178, 5803-5805, Aono, R., Tsukagoshi, N. & Yamamoto, M. (1998) J. Bacteriol. 180, 938-944). Changes in the expression of outer membrane proteins (e.g. increased OmpX, and decreased OmpF and LamB) have also been reported in E. coli marR mutants and wild type strains over-expressing MarA (Aono, R., Tsukagoshi, N. & Yamamoto, M. (1998) J. Bacteriol. 180, 938-944). MarA expression is shown herein to increase the transcription of both tolC and ompX (Table 3). Although a decrease in the levels of ompF, was observed, there was no evidence for a similar decrease in lamB expression, suggesting that LamB may not be the underproduced protein identified in the earlier study (Aono, R., Tsukagoshi, N. & Yamamoto, M. (1998) J. Bacteriol. 180, 938-944).

Transcription of the previously identified mlrl (b1451) and mlr2 (b0603) genes (Seoane, A. S. & Levy, S. B. (1995) J Bacteriol. 177, 530-535) was increased in the mar expression strain in two experiments, but appeared to be unaffected in a third experiment, so they were not included in Table 3. Expression of the slp gene, previously described as repressed by MarA (Seoane, A. S. & Levy, S. B. (1995) J. Bacteriol. 177, 530-535) was so low that any mar-mediated changes would have been difficult to detect. This latter observation may reflect these experiments being performed on cells in mid-logarithmic phase while slp is a stationary phase inducible gene. Since the identity of the two mar-responsive genes soi-17 and soi-19 (Greenberg, J. T., Chou, J. H., Monach, P. A. & Demple, B. (1991) J. Bacteriol. 173, 4433-4439) remains to be determined, their differential expression could not be confirmed by the macroarrays analysis.

### Example 3

Relationship between soxRS and mar regulons. SoxS is the ractivator of the soxRS regulon (Demple, B. (1996) Gene 179, 53-57), which mediates a cellular response to oxidative stress, and, like MarA, is a member of the XylS/AraC of transcriptional activators (Gallegos, M.-T., Schleif, R., Bairoch). Many oxidative stress genes, that are known to respond to SoxS, are also responsive to MarA (Jair, K.-W., Martin, R. G., Rosner, J. L., Fujita, N., Ishihama, A. & Wolf, J. R. E. (1995) J. Bacteriol 177, 7100-7104, Miller, P. F., Gambino, L. F., Sulavik, M. C. & Gracheck, S. J. (1994) Antimicrob. Agents Chemother. 38, 1773-1779). Conversely, SoxS is able to confer a Mar phenotype via activation of genes that are under the control of MarA (Ariza, R. R., Cohen, S. P., Bachbawat, N., Levy, S. B. & Demple, B. (1994) J. Bacteriol. 176, 143-148, Greenberg, J. T., Chou, J. H., Monach, P. A. & Demple, B. (1991) J. Bacteriol. 173, 4433-4439). Genes known to be regulated directly or indirectly by both the MarA and SoxS regulators include zwf; fpr, fumC., micF, nfo, inaA, soda and acrA (Ariza, R. R., Cohen, S. P., Bachbawat, N., Levy, S. B. & Demple, B. (1994) J. Bacteriol. 176, 143-148, Greenberg, J. T., Chou, J. H., Monach, P. A. & Demple, B. (1991) J. Bacteriol. 173, 4433-4439, Jair, K.-W., Martin, R. G., Rosner, J. L., Fujita, N., Ishihama, A. & Wolf, J. R. E. (1995) J. Bacteriol 177, 7100-7104, Rosner, J. L. & Slonczewski, J. L. (1994) J. Bacteriol. 176, 6262-6269, Liochev, S. 1. & Fridovich, I. (1992) Proc. Natl. Acad. Sci. USA 89, 5892-5896, Ma, D., Alberti, M., Lynch, C., Nikaido, H. & Hearst, J. E. (1996) Mol. Microbiol. 19, 101-112). The positive regulation of zwf fumC, acrA, inaA and sodA by mar, and also the down-regulation of ompF is confirmed by these results. However, although binding of MarA to nfo and fpr was shown in cell-free studies (Jair, K.-W., Martin, R. G., Rosner, J. L., Fujita, N., Ishihama, A. & Wolf, J. R. E. (1995) J. Bacteriol 177, 7100-7104), no significant change in expression of these two genes was detected using the experimental conditions employed here.

Other findings revealed further overlap between the mar and soxRS regulons. The levels of aconitase (acnA), GTP cyclohydrolase II (ribA) genes, and the major oxygen insensitive nitroreductase (nfsA/mdaA), previously known to be under the control of soxRS (Gruer, M. J. & Guest, J. R. (1994) Microbiology 140, 2531-2541, Koh, Y. S., Chung, W-H., Lee, J.-H. & Roe, J.-H. (1999) Mol. Gen. Cent., 374-380, Liochev, S.1., Hausladen, A. & Fridovich, I. (1999) Proc. Natl. Acad. Sci. USA 96, 3537-3539), were observed to be increased in mar-expressing strains (Table 3). While NfsA was shown to be the major isoenzyme affected by paraquat, the oxygen sensitive NAD(P)H nitroreductase B, nfnB (also designated nfsB), was shown to be slightly induced (Liochev, S.1., Hausladen, A. & Fridovich, I. (1999) Proc. Natl. Acad. Sci. USA 96, 3537-3539). nfnB, like nfsA, is under the positive control of mar (Table 3).

nfsA was initially designated mdaA (modulator of drug activity), as one of two genes associated with bacterial resistance to tumoricidal compounds (Chatterjee, P. K. & Sternberg, N. L. (1995) Proc. Natl. Acad. Sci. USA 92, 8950-8954). The other gene, designated mdaB, was also found to be affected by mar (Table 3). Information about mdaB is very limited, and its function remains unknown. These findings provide suggestive evidence for a putative physiological role in protection against environmental stresses.

The exact mechanisms for the overlapping regulation by MarA and SoxS are still poorly understood. Multiple antibiotic resistance encoded by the soxRS locus appeared partly dependent on an intact mar locus; strains overexpressing SoxS showed increased levels of mar RAB transcription (Miller, P. F., Gambino, L. F., Sulavik, M. C. & Gracheck, S. J. (1994) Antimicrob. Agents Chemother. 38, 1773-1779). On the other hand, other work showed that regulation of some genes by mar and by soxRS can occur through independent pathways, e.g. inaA (Rosner, J. L. & Slonczewski, J. L. (1994) J. Bacteriol. 176, 6262-6269). An effect of mar on soxRS has not been detected and no up-regulation of soxS expression by mar was observed. Therefore, MarA appears to operate independently of SoxS.

Rob, a MarA/SoxS homologue, is also able to bind to promoters of genes belonging to the mar-regulon and overexpression of this protein leads to multiple antibiotic resistance and organic solvent tolerance in E. coli (Ariza, R. R., Li, Z., Ringstad, N. & Demple, B. (1995) J. Bacteriol. 177, 1655-1661, Jair, K. W., Yu, X., Skarstad, K., Thony, B., Fujita, N., Ishihama, A. & Wolf, R. E. J. (1996) J. Bacteriol. 178, 2507-2513). No substantial change in expression of rob by MarA was found.

### EXAMPLE 4

mar regulation of operons and co-transcribed units. Some of the mar-regulated genes were clustered in discrete regions, as part of documented or predicted operons (Blattner, F. R., Plunkett, G. I. I. I., Bloch, C. A., Pema, N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462) (FIG. 2). Interestingly, considerable variability in the levels of expression of different genes from the same operon was observed, and therefore only some of these genes were eligible for listing in Table 3. For example, the fold increase in expression of the three genes in the tryptophanase operon (tnaLAB; 83.8 min) was 1.7 for tnaL and 8.1 for tnaA (averaged values), while tnaB was unclear; it gave background values in one experiment, but was clearly up-regulated in the other two experiments.

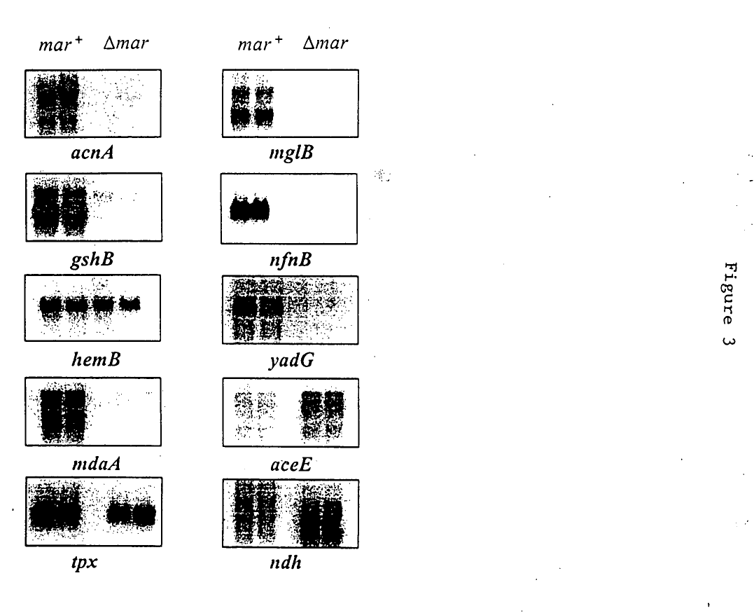

Differential expression of genes within mar-regulated operons could arise as result of other factors besides regulation of transcriptional initiation, e.g. differences in mRNA stability or the presence of regulatory secondary structures in the intercistronic regions of the operon. For example, the β-methylgalactoside (mgl) transport operon is composed of three Orfs, mglBAC. Northern analysis showed the presence of two transcripts, a polycistronic mglBAC mRNA and a smaller transcript which corresponds to the first gene in the operon, mglB (Hogg, R. W., Voelker, C. & von Carlowitz, I. (1991) Mol Cen. Genet. 229, 453-459). This finding was suggested to result from 3′-5′ degradation of the larger mRNA, and from protection of the smaller transcript against nucleases by a repetitive extragenic palindrome sequence located at its 3′ end. In agreement, these findings showed the smaller transcript at a much higher level than the larger one (FIG. 3). In FIG. 3, eight genes up-regulated by mar: acnA, gshB, hemB, mdaA, tpx, mglB, nfnB and yadG, and 2 genes down-regulated by mar: aceE and ndh, were selected from those listed in Table 3. Samples were prepared and run in duplicate from mar-expressing (mar⁺) and mar-deleted (Δmar) cells. RNA samples were transferred to nylon membranes and hybridized to ³²P-labeled PCR amplified probes of the genes in study

The only members of the mar regulon which appear to have a paralog in the E. coli genome are acrA, pflB, ompF, marA and mtr (http:/www.genetics.wisc.edu/). However, with the possible exception of mtr vs. tnaB, none of the paralogs for these genes was identified as being regulated by mar, and therefore artifacts of cross-hybridization with other genes sharing substantial sequence homology (Richmond, C. S., Glasner, J. D., Mau, R., Jin, H. & Blattner, F. R. (1999) Nucleic Acids Res. 27, 38213835) do not appear to account for the observed findings.

Mar regulation of neighboring genes which are not part of previously documented operons was also observed (Tables 3 and FIG. 2). Up-regulation of gshB (min 66.6) expression by mar was routinely observed; moreover, yggJ whose function remains unknown, and is located immediately upstream from gshB, and the Orf downstream from gshB, yqgE (b2948), were also up-regulated by MarA. There are only 13 bp between the end of yggJ and the beginning of gshB, and 37 bp between gshB and yqgE, which does not allow for the presence of promoter sequences in the respective intergenic regions. These results support the annotation of these three genes as a “predicted operon” (Blattner, F. R., Plunkett, G. I. I. I., Bloch, C. A., Perna, N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462).

Transcription of the gene ybjC, a small Orf immediately upstream from, nfsA, also seems to be up-regulated by MarA. A promoter sequence internal to ybjC and near its start codon has been proposed for nfsA (44). Thus, nfsA could be transcribed independently from this promoter but the resulting transcript would hybridize to both genes in the array. On the other hand, the E. coli genome sequence suggests that these two genes may form an operon (Blattner, F. R., Plunkett, G. 1.1. I., Bloch, C. A., Perna, N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462). The two genes downstream from nfsA, rimK and b0853, are also up regulated by MarA. A putative transcriptional terminator has been identified in the intergenic region of nfsA and rimK (Zenno, S., Koike, H., Kumar, A. N., Jayarman, R., Tanokura, M. & Saigo, K. (1996) J. Bacteriol. 178, 4508-4514). Nevertheless, a certain level of read-through transcription would explain the co-expression of this complex of genes.

### EXAMPLE 5

Relationship between the mar regulon and iron. Some of the genes regulated by MarA are associated with iron, e.g. hemB, fumC, fecA, acnA, sodA. The products of some of the genes contain iron-sulfur clusters, which play a major role in sensing O₂ and iron, and in regulatory functions (Beinert, H. & Kiley, P. J. (1999) Curr. Opin. Chem. Biol. 3, 152-157) (Ding, H. & Demple, B. (1998) Biochemistry 37, 17280-17286). Iron is an essential element for the bacterial cell (Earhart, C. F. (1996) in Escherichia coli and Salmonella: Cellular and Molecular Biology, eds. Neidhardt, F. C. (ASM Press, Washington, D.C.), pp. 1075-1090) and iron acquisition from the host is important in bacterial pathogenesis (Litwin, C. M. & Calderwood, S. B. (1993) Clin. Microbiol. Rev. 6, 137-149)(Mahan, M. J., Slauch, J. M. & Mekalanos, J. J. (1996) in Escherichia coli and Salmonella. Cellular and Molecular Biology, eds. Neidhardt, F. C. (ASM Press, Washington, D.C.), pp. 2803-2815). However, iron can also be harmful to the bacterial cell as it catalyzes the production of hydroxyl ions via the Fenton reaction, which may damage all cellular components and even lead to cell death (Zheng, M., Doan, B., Schneider, T. D. & Storz, G. (1999) J. Bacteriol. 181, 4639-4643).

Some genes known to be regulated by Fur (ferric uptake regulator), are also responsive to SoxS, MarA and other regulators e.g. acnA and soda (Cunningham, L., Gruer, M. J. & Guest, J. R. (1997) Microbiology 143, 3795-805) (Storz, G. & Imlay, J. A. (1999) Curr. Opin. Microbiol. 2, 188-194). This co-regulation would allow the cell to deal with the iron-associated oxidative stress and suggest a role for mar in bacterial pathogenesis.

### EXAMPLE 6

Northern blot analysis of selected genes. Ten newly identified mar-regulated genes, whose expression was either induced (tpx, acnA, mglB, mdaA, gshB, hemB, yadG and nfnB), or repressed (aceE and ndh) in the macroarrays were confirmed by Northern blot analysis. This showed changes in the expression of mono or polycistronic transcripts associated with the genes (FIG. 3). The magnitude of these changes, not unexpectedly, differed somewhat from that obtained for the macroarrays. Regulation of gshB, mdaA and aceE genes involved alteration in the levels of multiple transcripts as expected based on reported or predicted involvement of these genes in polycistronic elements (Blattner, F. R., Plunkett, G. I. I. I., Bloch, C. A., Perna, N., Burland, V., Riley, M., Collado-Vides, J., Glasner, J. D., Rode, C. K. M., G. F., Gregor, J., Davis, N. W., Kirkpatrick, H. A., Goeden, M. A., Rose, D. J., Mau, B. & Shao, Y. (1997) Science 277, 1453-1462), (Spencer, M. E. & Guest, J. R. (1985) Mol. Cen. Genet. 200, 145-154), (Quail, M. A., Ilaydon, D. J. & Guest, J. R. (1994) Mol. Microbiol. 12, 95-104).

The transcriptional activator MarA may control the expression of genes directly or indirectly. It could activate intermediate activator or inhibitor regulatory proteins which then could up- or down-regulate the expression of other genes in the regulon. A case in point is the mar-regulation of ompF mentioned earlier (Cohen, S. P., McMurry, L. M. & Levy, S. B. (1988) J. Bacteriol. 170, 5416-5422). MarA activates micF, an antisense RNA which negatively affects the translation of ompF, leading to decreased outer membrane porin OmpF (Cohen, S. P., Hachler, H. & Levy, S. B. (1993) J. Bacteriol. 175, 1484-1492, Cohen, S. P., McMurry, L. M., I-looper, D. C., Wolfson, J. S. & Levy, S. B. (1989) Antimicrob. Agents Chemother. 33, 1318-1325). Furthermore, transcriptional activators can act also as repressor proteins, depending on the position of the regulator binding site at the exclusive zone of repression (Gralla, J. D. & Collado-Vides, J. (1996) in Escherichia coli and Salmonella. Cellular and MolecularBiology, eds. Neidhardt, F. C. (ASM Press, Washington, D.C.), pp. 1232-1245).

Only those genes whose expression trends were consistent in three experiments are reported here. It is therefore likely that the size of the mar regulon is under estimated. Some of the genes containing putative marboxes in their promoter regions (Martin, R. G., Gillette, W. K., Rhee, S. & Rosner, J. L. (1999) Mol. Microbiol. 34, 431-441) were not shown under the conditions used here to be part of the mar regulon. Moreover, a large number of genes was expressed at background level or responded to mar expression with small changes that were below the threshold applied in this study and therefore were not included. Under a different set of experimental conditions, such as using cells in a different stage of the growth phase, or grown in different media, it is possible that the magnitude of these changes will increase, or new genes will be affected, justifying inclusion in the mar regulon. Certainly small and transient changes in gene expression could have important implications in the cell's response to external stresses. Differences observed in global expression analysis between experiments have been seen and extensively addressed by other authors (Richmond, C. S., Glasner, J. D., Mau, R., Jin, H. & Blattner, F. R. (1999) Nucleic Acids Res. 27, 38213835) (Tao, H., Bausch, C., Richmond, C., Blattner, F. R. & Conway, T. (1999) J. Bacteriol. 181, 6425-6440). Among other factors the authors observed that the signal intensity of some genes was significantly different between experiments when using different batches of RNA. This problem was addressed in part by performing the study in triplicate. Trends detected by the gene array method must, therefore, be analyzed by other available molecular and biochemical techniques, such as Northern blot analysis and promoter fusion studies.

TABLE 3
Genes identified as part of the mar regulon using the E. coli Panorama
gene arrays.

| Gene name | Product* | MarA regulation |
|---|---|---|
| | Up-regulated genes | |
| acnA | Aconitate hydrase 1 | 2.7/5.9 |
| acrA | Acridine efflux pump | 1.9/2.3 |
| aldA | Aldehyde dyhydrogenase, NAD-linked | 7.4/3.2 |
| b0447 | Putative LRP-like transcriptional | 3.5/4.4 |
| | regulator | |
| b0853 | Putative sensory transduction regulator | 1.4/4.2 |
| b1448 | Putative resistance protein | 1.8/2.3 |
| b2889 | Putative enzyme | 2.5/5.6 |
| b2948 | Orf; hypothetical protein | 1.4/2.5 |
| cobU | Cobinamide kinase/cobinamide | 1.6/2.2 |
| | phosphate guanylytransferase | |
| fumC | Fumarase C = fumarase hydratase Class | 2.5/2.9 |
| | II; isoenzyme | |
| galK | galactokinase | 1.5/2.0 |
| galT | Galactose-1-phosphate | 2.5/2.4 |
| | uridylyltransferase | |
| gatA | Galactitol-specific enzyme IIA of | 2.0/1.8 |
| | phosphotransferase system | |
| gatC | PTS system galactitol-specific enzyme | 3.4/1.6 |
| | IIC | |
| gltA | Citrate synthase | 2.1/1.9 |
| gshB | Glutathione synthetase | 3.5/5.7 |
| hemB | 5-aimnolevulinate | 5.7/5.1 |
| | dehydratase = porphobilinogen synthase | |
| inaA | pH-inducible protein involved in stress | 5.0/20.2 |
| | response | |
| map | Methionine aminopeptidase | 1.7/2.1 |
| marA | Multiple antibiotic resistance; | 24.0/46.6 |
| | transcriptional activator of defense | |
| | systems | |
| marB | Multiple antibiotic resistance protein | 7.5/16.3 |
| marR | Multiple antibiotic resistance protein; | 15.9/46.3 |
| | repressor of mar operon | |
| mdaA | Modulator of drug resistance A | 3.8/8.2 |
| mdaB | Modulator of drug resistance B | 5.5/8.2 |
| mglB | Galactose-binding transport protein; | 5.3/2.6 |
| | receptor for galactose taxis | |
| mtr | Tryptophan-specific transport protein | 1.3/2.2 |
| nfnB | Oxygen-insensitive NAD(P)H | 12.4/20.1 |
| | nitroreductase | |
| ompX | Outer membrane protein X | 1.6/2.1 |
| pflB | Formate acetyltransferase 1 | 2.1/2.2 |
| pgi | Glucose-6-phosphate isomerase | 2.4/2.1 |
| ribA | GTP cyclohydrolase II | 1.1/2.2 |
| ribD | Bifunctional pyrimidine | 1.7/2.5 |
| | deaminase/reductase in pathway of | |
| | riboflavin synthesis | |
| rimK | Ribosomal protein S6 modification | 1.6/3.0 |
| | protein | |
| sodA | Superoxide dismutase, manganese | 7.0/4.6 |
| slA_2 | PTS system, glucitol/sorbitol-specific | 3.0/2.0 |
| | IIB component and second of two IIC | |
| | component | |
| tnaA | Tryptophanase | 7.9/8.4 |
| tnaL | Tryptophanase leader peptide | 1.3/2.1 |
| tolC | Outer membrane channel; specific | 3.1/2.8 |
| | tolerance to colicin E1; segregation of | |
| | daughter chromosomes | |
| tpx | Thiol peroxidase | 2.1/1.6 |
| yadG | Putative ATP-binding component of a | 9.2/11.2 |
| | transport system | |
| yadH | Orf; hypothetical protein | 1.9/2.7 |
| ybjC | Orf, hypothetical protein | 6.7/17.4 |
| ydeA | Putative resistance/regulatory protein | 1.9/3.9 |
| yfaE | Orf, hypothetical protein | 2.5/5.9 |
| yggJ | Orf, hypothetical protein | 3.1/4.2 |
| yhbW | Putative enzyme | 10.6/6.5 |
| zwf | Glucose-6-phosphate dehydrogenase | 2.7/1.8 |
| | Down-regulated genes | |
| accB | Acetyl-CoA carboxylase, BCCP | 2.2/2.0 |
| | subunit; carrier of biotin | |
| aceE | Pyruvate dehydrogenase | 6.1/5.2 |
| | (decarboxylase component) | |
| aceF | Pyruvate dehydrogenase (dihydro | 5.1/4.1 |
| | lipoltransacetylase component) | |
| ackA | Acetate kinase | 1.8/2.6 |
| b0357 | Putative alpha helix chain | 3.2/2.2 |
| b2530 | Putative aminotransferase | 1.2/2.3 |
| b3469 | Zinc-transporting ATPase | 1.6/2.2 |
| fabB | 3-oxoacyl-[acyl-carrier-protein] | 2.6/3.1 |
| | synthase I | |
| fecA | citrate-dependent iron transport, Outer | 2.5/2.8 |
| | membrane receptor | |
| glpD | Sn-glycerol-3-phosphate | 1.4/2.1 |
| | dehydrogenase (aerobic) | |
| guaB | IMP dehydrogenase | 2.9/2.3 |
| ndh | Respiratory NADH dehydrogenase | 5.8/3.8 |
| ompF | Outer membrane protein 1a (Ia; b; F) | 2.7/3.0 |
| purA | Adenylosuccinate synthetase | 2.1/2.1 |
| rplE | 50S ribosomal subunit protein L5 | 3.5/2.0 |

*Information about individual genes was obtained through the E. coli K-12 genome project Web page (http://www.genetics.wisc.edu/). mar regulation corresponds to ratios of gene expression between experimental and control samples for the up-regulated and the opposite for the down-regulated genes, obtained from two independent experiments.

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

1. A method for identifying compounds that modulate an NIMR polypeptide activity comprising:
contacting an NIMR polypeptide with a test compound under conditions which allow interaction of the compound with the polypeptide;
determining the ability of the test compound to modulate the activity of an NIMR polypeptide; and
selecting those compounds that modulate the activity of the NIMR polypeptide to thereby identify compounds that modulate NIMR polypeptide activity.

2. The method of claim 1, wherein the NIMR polypeptide is selected from the group consisting of: b0357, b0447, b0853, b1448, b2530, b2889, b2948, b3469, mdaB, yadG, yadH, ybjC, yfaE, yggJ, and yhbW.

3. The method of claim 1, wherein the NIMR polypeptide activity comprises promoting the ability of a microbe to resist an environmental challenge.

4. The method of claim 3, wherein the NIMR polypeptide is selected from the group consisting of: aceG, ackA, aldA, cobU, fabB, fecA, galK, galT, gatA, gatC, glpD, gltA, gshB, guaB, hemB, map, mglB, mtr, ndh, nfnB, pflB, pgi, purA, ribD, rimK, rplE, srlA_{—}2, tnaA, tnaL, tpx, acnA, mdaA, ribA, and ydeA.

5. The method of claim 1, wherein the NIMR polypeptide activity comprises promotion of microbial virulence.

6. The method of claim 5, wherein the NIMR polypeptide is selected from the group consisting of: aceG, ackA, aldA, cobU, fabB, fecA, galK, galT, gatA, gatC, glpD, gltA, gshB, guaB, hemB, map, mglB, mtr, ndh, nfnB, pflB, pgi, purA, ribD, rimK, rplE, srlA_{—}2, tnaA, tnaL, tpx, acnA, mdaA, ribA, and ydeA.

7. The method of any of claims 1, 3, or 5 wherein said step of determining comprises measuring the efflux of the test compound or a marker compound from the cell.

8. The method of any of claims 1, 3, or 5 wherein said step of determining comprises measuring the ability of the microbe to grow or remain viable in the presence of the environmental challenge.

9. The method of any of claims 1, 3, or 5 wherein the NIMR polypeptide is present in a microbial cell.

10. The method of claim 9, wherein the NIMR polypeptide is heterologous to the cell in which it is present.

11. A method for identifying compounds that modulate an NIMR polypeptide activity comprising:
contacting an NIMR polypeptide with a test compound under conditions which allow interaction of the compound with the polypeptide;
determining the ability of the test compound to modulate the expression of an NIMR polypeptide; and
selecting those compounds that modulate the expression of the NIMR polypeptide to thereby identify compounds that modulate NIMR polypeptide activity.

12. The method of claim 11, wherein the NIMR polypeptide is selected from the group consisting of: b0357, b0447, b0853, b1448, b2530, b2889, b2948, b3469, mdaB, yadG, yadH, ybjC, yfaE, yggJ, and yhbW.

13. The method of claim 11, wherein the NIMR polypeptide is selected from the group consisting of: aceG, accB, acef, ackA, aldA, cob U, fabB, feca, galk, galt gatA, gatC, glpD, gltA, gshB, guaB, hemB, map, mglB, mtr, ndh, nfnb, pflB, pgi, purA, ribD, rimK, rplE, srlA_{—}2, tnaA, tnaL, tpx, acnA, mdaA, ribA, and ydeA.

14. The method of any one of claims 12 or 13, wherein the step of measuring comprises measuring the amount of RNA produced by the cell.

15. The method of any one of claims 12 or 13, wherein the step of measuring comprises measuring the amount or activity of a reporter gene product produced by the cell.

16. The method of claim 15 wherein the step of measuring comprises detecting the ability of an antibody to bind to the reporter gene product.

17. The method of any of claims 1, 3, or 5 wherein the NIMR polypeptide is present in a cell free system.

18. The method of claim 17, wherein the step of determining comprises measuring the ability of the compound to bind to the NIMR polypeptide.

19. A method for decreasing the virulence of a microbe comprising exposing the microbe to an environmental challenge and to an agent that modulates the activity of an NIMR polypeptide.

20. A method for reducing the marA mediated transcription of an NIMR gene comprising exposing the microbe to an environmental challenge and to an agent that modulates the activity of an NIMR polypeptide.

21. A method for identifying compounds that modulate activity of an NIMR polypeptide in a microbe comprising:
contacting an isolated NIMR nucleic acid molecule with a test compound under conditions which allow interaction of the compound with the nucleic acid molecule;
determining the ability of the test compound to bind to the isolated NIMR nucleic acid molecule; and
selecting those compounds that bind to the NIMR nucleic acid molecule to thereby identify compounds that modulate activity of an NIMR polypeptide.

22. The method of claim 21, wherein the NIMR polypeptide is selected from the group consisting of: b0357, b0447, b0853, b1448, b2530, b2889, b2948, b3469, mdaB, yadG, yadH, ybjC, yfaE, yggJ, and yhbW.

23. The method of claim 21, wherein the NIMR polypeptide activity comprises promoting the ability of a microbe to resist an environmental challenge.

24. The method of claim 22, wherein the NIMR polypeptide is selected from the group consisting of: accB, aceF, aceG, acka, aldA, cobU, fabB, feca, galK, galt, gatA, gatC, glpD, gltA, gshB, guaB, hemB, map, mglB, mtr, ndh, nfnB, pflB, pgi, purA, ribD; rimK, rplE, srlA_{—}2, tnaA, tnaL, tpx, acnA, mdaA, ribA, and ydeA.

25. The method of claim 19, wherein the NIMR polypeptide activity comprises promotion of the virulence of a microbe.

26. The method of claim 25, wherein the NIMR polypeptide is selected from the group consisting of: aceG, ackA, aldA, cobU, fabB, fecA, galK, galt, gatA, gatC, glpD, gltA, gshB, guaB, hemB, map, mglB, mtr, ndh, nfnB, pflB, pgi, purA, ribD, rimK, rplE, srlA_{—}2, tnaA, tnaL, tpx, acna, mdaA, ribA, and ydeA.

27. The method of claim 21, wherein the environmental challenge is an antibiotic compound.

28. The method of claim 21, wherein the environmental challenge is non-antibiotic compound.

29. The method of claim 28, wherein the non-antibiotic compound is a candidate disinfectant or antiseptic compound.

30. A vaccine comprising at least one NIMR nucleic acid molecule or an NIMR polypeptide and a pharmaceutically acceptable carrier.

31. A composition comprising at least one compound that modulates the activity of an NIMR polypeptide and at least one antibiotic.

32. A composition comprising at least one compound that modulates the activity of an NIMR polypeptide and at least one non-antibiotic compound.

33. A method for reducing the virulence of a microbe in a subject suffering from a microbial infection comprising administering at least one NIMR modulating agent to the subject such that the virulence of the microbe is reduced.

34. A method for treating a microbial infection in a subject comprising administering at least one NIMR modulating agent to the subject such that the infection is treated.

35. A method for reducing the infectivity of a microbe on a surface comprising contacting the microbe with at least one NIMR modulating agent such that the infectivity of the microbe is reduced.

36. The method of any one of claims 33, 34, or 35, wherein the microbe is a gram positive bacteria.

37. The method of any one of claims 33, 34, or 35, wherein the microbe is a gram negative bacteria.

38. The method of any one of claims 33, 34, or 35, wherein the microbe is an acid fast bacteria.
